(19) United States
(12) Patent Application Publication
(10) Pub. No.: US 2025/0230202 A1
(43) Pub. Date: Jul. 17, 2025

(54) USE OF PROMYELOCYTIC LEUKEMIA 1 (PML-1) PROTEIN IN PREPARATION OF DRUG FOR INHIBITING CYTOKINE STORM

(71) Applicant: Chengdu Fudai Biomedical Co., Ltd., Chengdu City (CN)

(72) Inventors: Fubing SHEN, Chengdu City (CN); Dai SHEN, Chengdu City (CN); Mengling LIU, Chengdu City (CN)

(73) Assignee: Chengdu Fudai Biomedical Co., Ltd., Chengdu City (CN)

(21) Appl. No.: 18/411,600

(22) Filed: Jan. 12, 2024

(51) Int. Cl. C07K 14/435; A61K 38/00; A61P 29/00; C12N 15/86

(52) U.S. Cl. CPC C07K 14/435; A61P 29/00; C12N 15/86; A61K 38/00; C12N 2740/15043

(57) ABSTRACT

The present disclosure provides use of a promyelocytic leukemia 1 (PML-1) protein in preparation of a drug for inhibiting a cytokine storm, and belongs to the technical field of biomedicine. The present disclosure further provides use of a PML-1 protein and/or a product expressing the PML-1 protein in preparation of a drug for inhibiting a cytokine storm. In the present disclosure, the PML-1 protein and/or the product expressing the PML-1 protein can significantly inhibit the expression of proteins TAB1, TAK1, and p-TAK1 and inhibit inflammatory cytokines TNF-α, IL-1β, MIP-1α, IL-6, IL-8, and MCP-1 in an inflammatory signaling pathway, thereby inhibiting the cytokine storm. The PML-1 protein can be directly used for the treatment of inflammations and related diseases. Compared with traditional chemical anti-inflammatory drugs, the protein shows more significant curative effect, stronger specificity, lower toxicity, smaller side effects, and clearer biological functions, exhibiting broad application prospects.

### SEQUENCE LISTING

The instant application contains an electronic sequence listing. The contents of the electronic sequence listing H2936376.xml; size: 37,321 bytes; and Date of Creation: Feb. 28, 2024, is herein incorporated by reference in its entirety.

### TECHNICAL FIELD

The present disclosure belongs to the technical field of biomedicine, and specifically relates to use of a promyelocytic leukemia 1 (PML-1) protein in preparation of a drug for inhibiting a cytokine storm.

### BACKGROUND

“Cytokine storm”, as a pathological immune phenomenon that has attracted much attention in recent years, refers to a pathological process in which the body's immune cells, especially macrophages, are activated by bacterial or viral infections and then express a large number of pro-inflammatory cytokines, triggering a severe inflammatory response. When the body's anti-inflammatory immune response cannot attenuate the inflammatory trend, a severe pathological damage may be caused to the organs at infection sites to induce multiple organ failure, leading to the death of patients. The “cytokine storm” was first reported in a 1993 article on graft-versus-host disease, and appeared frequently in reports on cytomegalovirus, Epstein-Barr virus-related hemophagocytic lymphohistiocytosis, group A streptococci, influenza viruses, H5N1 influenza virus infection, and SARS-CoV infection. The most eye-catching one was the cytokine storm of coronavirus pneumonia 2019 (COVID-19). In addition to acute infections, cytokine storms are also a common endpoint of various diseases such as autoimmune diseases (rheumatoid and systemic lupus erythematosus), tumors, and AIDS.

Early literatures have reported that promyelocytic leukemia (PML) is pro-inflammatory, and there are also reports about that cytoplasmic promyelocytic leukemia (cPML) can regulate biological functions of the PML by regulating nuclear promyelocytic leukemia (nPML). The PML includes different isomers, where PML-2, PML-3, PML-4, PML-5, and PML-6 are nuclear isomers; PML-7 and PML-14, which lack nuclear localization signals, are cytoplasmic isoforms; while PML-1 can shuttle between the nucleus and cytoplasm. Different isomers show different structures and different distributions in cells, resulting in different functions. However, there is a lack of relevant reports on an anti-inflammatory effect of PML-1 on the cytokine storm.

### SUMMARY

In view of the deficiencies in the prior art, an objective of the present disclosure is to provide use of a PML-1 protein and/or a product expressing the PML-1 protein in preparation of a drug for inhibiting an inflammatory cytokine storm. In the present disclosure, the PML-1 protein and/or the product expressing the PML-1 protein are used to inhibit the inflammatory cytokine storm.

The objective of the present disclosure is achieved by the following technical solutions:

The present disclosure provides use of a PML-1 protein and/or a product expressing the PML-1 protein in one or more of the following items (1) to (5):
(1) preparing a drug for inhibiting a cytokine storm;
(2) preparing an anti-inflammatory drug;
(3) preparing a drug for treating a disease with the cytokine storm as a main pathological feature;
(4) preparing a drug for regulating an inflammatory signaling pathway; and
(5) preparing a drug for regulating an inflammatory cytokine; wherein
the PML-1 protein has an amino acid sequence shown in SEQ ID NO: 3.

Preferably, the disease is one or more selected from the group consisting of pneumonia, an autoimmune disease, a tumor, AIDS, and a neurodegenerative disease; and
the inflammatory cytokine is one or more selected from the group consisting of TNF-α, IL-1β, IL-6, IL-8, MIP-1α, and MCP-1.

Preferably, the PML-1 protein is encoded with a nucleotide sequence shown in SEQ ID NO: 1 or SEQ ID NO: 15.

Preferably, the product includes a recombinant vector.

Preferably, the recombinant vector is selected from the group consisting of a lentiviral recombinant vector and a mammalian recombinant vector.

The present disclosure further provides a recombinant vector expressing a PML-1 protein, including a base vector and a nucleotide sequence encoding the PML-1 protein; where
the base vector is selected from the group consisting of a lentiviral base vector and a mammalian base vector.

Preferably, the lentiviral base vector uses an elongation factor 1 alpha (EF1A) as a promoter; and
the mammalian base vector comprises pcDNA3.4.

The present disclosure further provides a preparation method of a PML-1 protein and/or a product expressing the PML-1 protein, including expressing the recombinant vector.

Preferably, when the recombinant vector is a lentiviral recombinant vector, the lentiviral base vector and a helper plasmid are co-expressed; and
the helper plasmid is selected from the group consisting of SL3, SL4, and SL5.

The present disclosure further provides a PML-1 protein and/or a product expressing the PML-1 protein prepared by the preparation method.

Beneficial effects:
The PML-1 protein and/or product expressing the PML-1 protein can inhibit the synthesis and expression of cytokines by directly inhibiting a signaling pathway of Toll-like receptors (TLRs), thereby inhibiting the inflammatory cytokine storm. In the present disclosure, the PML-1 protein and/or the product expressing the PML-1 protein can particularly significantly inhibit the expression of proteins TAB1, TAK1, and p-TAK1 and significantly inhibit inflammatory cytokines TNF-α, IL-1β, MIP-1α, IL-6, IL-8, and MCP-1 in an inflammatory signaling pathway, thereby inhibiting the cytokine storm. In addition, the PML-1 protein and/or product expressing the PML-1 protein can be directly used for the treatment of inflammations and related diseases. Compared with traditional chemical anti-inflammatory drugs, the protein shows more significant curative effect, stronger specificity, lower toxicity, smaller side effects, and clearer biological functions, exhibiting broad application prospects.

Furthermore, the PML-1 protein and/or product expressing the PML-1 protein of the present disclosure are particularly suitable for the treatment of acute pneumonia such as COVID-9 and influenza, in which cytokine storm and inflammatory storm are the main pathological features.

Other diseases with the cytokine storm as an endpoint, such as systemic lupus erythematosus and rheumatoid arthritis, are also desirable indications of the protein. The protein can also be used in neurodegenerative diseases caused by bacterial and viral infections, including Alzheimer's disease, Parkinson's disease, multiple sclerosis, and Huntington's disease.

### BRIEF DESCRIPTION OF THE DRAWINGS

To illustrate the examples of the present disclosure or the technical solutions in the prior art more clearly, the accompanying drawings required in the examples will be briefly introduced below.

FIG. 1 shows a flow chart for constructing a PML-1 overexpression vector in Implementation Examples I and II;

FIG. 2 shows a complete structure of a SC493-1 plasmid;

FIG. 3 shows enzymatic digestion identification results of a recombinant plasmid in Implementation Example I;

FIG. 4 shows a lysis curve of detecting an expression level of the PML-1 by qPCR in Example 2 of Implementation Example I;

FIG. 5 shows fluorescence detection results of a lentivirus expressing the PML-1 in Implementation Example I, at a scale bar of 100 μm;

FIG. 6 shows Western blotting detection results of the lentivirus expressing the PML-1;

FIG. 7 shows Western blotting detection results in Example 4 of Implementation Example I;

FIGS. 8A-C show regulation results of the PML-1 on cytokines in an in vitro cell experiment in Example 5 of Implementation Example I;

FIG. 9 shows results of protein regulation of a TLR4 signaling pathway by PML-1 in an in vivo experiment in Example 6 of Implementation Example I;

FIGS. 10A-C show regulation results of the PML-1 on cytokines in an in vivo cell experiment in Example 6 of Implementation Example I;

FIGS. 11A-B show pathological sections and lung injury scoring results of PML-1-treated mice with pneumonia in Implementation Example I;

FIG. 12 shows a structure of an overexpression vector in Example 1 of Implementation Example II;

FIG. 13 shows enzymatic digestion identification results in Example 1 of Implementation Example II;

FIG. 14 shows a detection chart of the PML-1 overexpression plasmid extracted by agarose gel analysis in Example 2 of Implementation Example II;

FIG. 15 shows SDS-PAGE analysis results of the PML-1 recombinant protein in Example 2 of Implementation Example II;

FIGS. 16A-B show Western blotting band position and SDS-PAGE detection results of the PML-1 recombinant protein in Example 2 of Implementation Example II;

FIG. 17 shows detection results of an anti-inflammatory effect of the PML-1 recombinant protein in Example 3 of Implementation Example II;

FIGS. 18A-F show detection results for an influence of the PML-1 recombinant protein on inflammatory cytokines in Example 4 of Implementation Example II;

FIGS. 19A-E show detection results of the inflammatory cytokines in Example 5 of Implementation Example II;

FIGS. 20A-B show results of treating pneumonia in mice with the PML-1 recombinant protein in Implementation Example I; and

FIG. 21 shows a schematic diagram of the technical solution in the present disclosure.

### DETAILED DESCRIPTION OF THE EMBODIMENTS

The present disclosure provides use of a PML-1 protein and/or a product expressing the PML-1 protein in one or more of the following items (1) to (5):
(1) preparing a drug for inhibiting a cytokine storm;
(2) preparing an anti-inflammatory drug;
(3) preparing a drug for treating a disease with the cytokine storm as a main pathological feature;
(4) preparing a drug for regulating an inflammatory signaling pathway; and
(5) preparing a drug for regulating an inflammatory cytokine; wherein

In the present disclosure, the PML-1 protein has an amino acid sequence shown in SEQ ID NO: 3 and an accession number in NCBI of P29590, specifically:

MEPAPARSPRPQQDPARPQEPTMPPPETPSEGRQPSPSPSPTERA
PASEEEFQFLRCQQCQAEAKCPKLLPCLHTLCSGCLEASGMQCPI
CQAPWPLGADTPALDNVFFESLQRRLSVYRQIVDAQAVCTRCKES
ADFWCFECEQLLCAKCFEAHQWFLKHEARPLAELRNQSVREFLDG
TRKTNNIFCSNPNHRTPTLTSIYCRGCSKPLCCSCALLDSSHSEL
KCDISAEIQQRQEELDAMTQALQEQDSAFGAVHAQMHAAVGQLGR
ARAETEELIRERVRQVVAHVRAQERELLEAVDARYQRDYEEMASR
LGRLDAVLQRIRTGSALVQRMKCYASDQEVLDMHGFLRQALCRLR
QEEPQSLQAAVRTDGFDEFKVRLQDLSSCITQGKDAAVSKKASPE
AASTPRDPIDVDLPEEAERVKAQVQALGLAEAQPMAVVQSVPGAH
PVPVYAFSIKGPSYGEDVSNTTTAQKRKCSQTQCPRKVIKMESEE
GKEARLARSSPEQPRPSTSKAVSPPHLDGPPSPRSPVIGSEVFLP
NSNHVASGAGEAEERVVVISSSEDSDAENSSSRELDDSSSESSDL
QLEGPSTLRVLDENLADPQAEDRPLVFFDLKIDNETQKISQLAAV
NRESKFRVVIQPEAFFSIYSKAVSLEVGLQHFLSFLSSMRRPILA
CYKLWGPGLPNFFRALEDINRLWEFQEAISGFLAALPLIRERVPG
ASSFKLKNLAQTYLARNMSERSAMAAVLAMRDLCRLLEVSPGPQL
AQHVYPFSSLQCFASLQPLVQAAVLPRAEARLLALHNVSFMELLS
AHRRDRQGGLKKYSRYLSLQTTTLPPAQPAFNLQALGTYFEGLLE
GPALARAEGVSTPLAGRGLAERASQQS.

In the present disclosure, the PML-1 protein is encoded with a nucleotide sequence of preferably 2,649 bp in length and preferably shown in SEQ ID NO: 1, specifically:

atggagcctgcacccgcccgatctccgaggccccagcaggacccc
gcccggccccaggagcccaccatgcctccccccgagaccccctct
gaaggccgccagcccagccccagccccagccctacagagcgagcc
cccgcttcggaggaggagttccagtttctgcgctgccagcaatgc
caggcggaagccaagtgcccgaagctgctgccttgtctgcacacg
ctgtgctcaggatgcctggaggcgtcgggcatgcagtgccccatc
tgccaggcgccctggcccctaggtgcagacacacccgccctggat
aacgtctttttcgagagtctgcagcggcgcctgtcggtgtaccgg
cagattgtggatgcgcaggctgtgtgcacccgctgcaaagagtcg
gccgacttctggtgctttgagtgcgagcagctcctctgcgccaag
tgcttcgaggcacaccagtggttcctcaagcacgaggcccggccc
ctagcagagctgcgcaaccagtcggtgcgtgagttcctggacggc
acccgcaagaccaacaacatcttctgctccaaccccaaccaccgc
acccctacgctgaccagcatctactgccgaggatgttccaagccg
ctgtgctgctcgtgcgcgctccttgacagcagccacagtgagctc
aagtgcgacatcagcgcagagatccagcagcgacaggaggagctg
gacgccatgacgcaggcgctgcaggagcaggatagtgcctttggc
gcggttcacgcgcagatgcacgcggccgtcggccagctgggccgc
gcgcgtgccgagaccgaggagctgatccgcgagcgcgtgcgccag
gtggtagctcacgtgcgggctcaggagcgcgagctgctggaggct
gtggacgcgcggtaccagcgcgactacgaggagatggccagtcgg
ctgggccgcctggatgctgtgctgcagcgcatccgcacgggcagc
gcgctggtgcagaggatgaagtgctacgcctcggaccaggaggtg
ctggacatgcacggtttcctgcgccaggcgctctgccgcctgcgc
caggaggagccccagagcctgcaagctgccgtgcgcaccgatggc
ttcgacgagttcaaggtgcgcctgcaggacctcagctcttgcatc
acccaggggaaagatgcagctgtatccaagaaagccagcccagag
gctgccagcactcccagggaccctattgacgttgacctgcccgag
gaggcagagagagtgaaggcccaggttcaggccctggggctggct
gaagcccagcctatggctgtggtacagtcagtgcccggggcacac
cccgtgccagtgtacgccttctccatcaaaggcccttcctatgga
gaggatgtctccaatacaacgacagcccagaagaggaagtgcagc
cagacccagtgccccaggaaggtcatcaagatggagtctgaggag
gggaaggaggcaaggttggctcggagctccccggagcagcccagg
cccagcacctccaaggcagtctcaccaccccacctggatggaccg
cctagccccaggagccccgtcataggaagtgaggtcttcctgccc
aacagcaaccacgtggccagtggcgccggggaggcagaggaacgc
gttgtggtgatcagcagctcggaagactcagatgccgaaaactcg
tcctcccgagagctggatgacagcagcagtgagtccagtgacctc
cagctggaaggccccagcaccctcagggtcctggacgagaacctt
gctgacccccaagcagaagacagacctctggttttctttgacctc
aagattgacaatgaaacccagaagattagccagctggctgcggtg
aaccgggaaagcaagttccgcgtggtcatccagcctgaagccttc
ttcagcatctactccaaggccgtgtccctggaggtggggctgcag
cacttcctcagctttctgagctccatgcgccgccctatcttggcc
tgctacaagctgtgggggcctggcctcccaaacttcttccgggcc
ctggaggacattaacaggctgtgggaattccaggaggccatctcg
ggcttcctggctgccctgcctctcatccgggagcgtgtgcccggg
gccagcagcttcaaactcaagaacctggcccagacctacctggcg
agaaacatgagcgagcgcagcgccatggctgccgtgctggccatg
cgtgacctgtgccgcctcctcgaggtctccccgggcccccagctg
gcccagcatgtctaccccttcagtagcctgcagtgctttgcctcc
ctgcagcccctggtgcaggcagctgtgctgccccgggctgaggcc
cgcctcctggccctacacaacgtgagcttcatggagctgctgagt
gcacaccgccgtgaccggcaggggggcctgaagaagtacagccgc
tatctaagcctgcagaccaccacgttgccccctgcccagcctgct
ttcaacctgcaggctctgggcacctactttgaaggcctgttggag
ggtccggcgctggcacgggcagaaggagtctccaccccacttgct
ggccgtggcttggcagagagggcctcccagcagagctga.

In the present disclosure, the PML-1 protein is encoded with a nucleotide sequence preferably shown in SEQ ID NO: 15, specifically:

GAATTCCCGCCGCCACCATGGAGCCAGCTCCAGCCAGAAGCCCTA
GACCTCAGCAGGACCCCGCCAGACCTCAGGAACCTACCATGCCTC
CTCCAGAGACACCTAGCGAGGGCAGACAGCCTTCTCCTAGCCCTT
CTCCTACAGAGAGAGCCCCAGCTAGCGAGGAAGAGTTCCAGTTCC
TCCGCTGCCAGCAGTGTCAGGCAGAAGCCAAGTGCCCTAAGCTGC
TGCCTTGCCTGCACACCCTCTGCAGTGGTTGTCTGGAGGCTAGCG
GCATGCAGTGTCCCATTTGCCAGGCTCCTTGGCCTCTGGGAGCAG
ATACCCCAGCCCTGGACAACGTGTTCTTCGAGAGCCTGCAGCGGA
GACTGAGCGTGTACAGACAGATCGTGGACGCTCAGGCCGTGTGTA
CACGCTGCAAGGAATCCGCCGACTTTTGGTGCTTCGAGTGCGAGC
AGCTCCTCTGCGCTAAGTGCTTCGAGGCCCACCAGTGGTTCCTGA
AGCACGAAGCCAGACCTCTGGCCGAACTGAGAAACCAGAGCGTGC
GAGAGTTTCTGGACGGCACCCGCAAGACCAACAACATCTTCTGCA
GCAACCCCAACCACAGAACCCCTACCCTGACCAGCATCTATTGCC
GGGGTTGCAGCAAGCCCCTGTGTTGCTCTTGCGCTCTGCTGGACA
GCAGCCACAGCGAACTCAAGTGCGACATCAGCGCCGAGATCCAGC
AGAGACAGGAGGAACTGGACGCCATGACACAGGCTCTGCAGGAAC
AGGATAGCGCCTTTGGAGCAGTGCACGCTCAGATGCACGCAGCAG
TGGGACAGCTGGGAAGAGCCAGAGCCGAAACAGAGGAGCTGATCA
GGGAGAGAGTGAGACAGGTGGTGGCTCACGTCAGAGCCCAGGAAA
GAGAGCTGCTGGAGGCAGTGGACGCTAGATACCAGCGGGACTACG
AGGAGATGGCCTCTAGACTGGGCAGACTGGACGCCGTGCTGCAGA
GAATCAGGACAGGAAGCGCCCTGGTGCAGCGCATGAAGTGTTACG
CTAGCGATCAGGAGGTGCTGGACATGCACGGATTCCTGAGGCAGG
CTCTCTGCAGACTGAGGCAGGAAGAGCCACAGTCTCTGCAGGCAG
CAGTGCGGACAGACGGCTTCGACGAGTTCAAGGTCCGCCTGCAGG
ATCTGAGCAGTTGCATCACCCAGGGAAAAGACGCCGCCGTGTCTA
AGAAGGCCTCTCCAGAAGCCGCCTCTACACCTAGGGACCCTATCG
ACGTGGATCTGCCAGAGGAGGCAGAGAGAGTGAAGGCTCAGGTGC
AGGCTCTGGGACTGGCAGAAGCTCAGCCTATGGCAGTGGTGCAGT
CAGTGCCAGGAGCTCATCCAGTGCCCGTGTACGCCTTCAGCATCA
AGGGACCTAGCTACGGCGAGGACGTGTCCAATACCACCACCGCCC
AGAAGCGCAAGTGTTCTCAGACCCAGTGCCCCCGGAAGGTCATCA
AGATGGAGAGCGAGGAGGGCAAGGAGGCTAGACTGGCTAGAAGCA
GCCCAGAGCAGCCTAGACCTAGCACAAGCAAGGCCGTGTCTCCTC
CTCACCTGGACGGACCTCCTTCTCCTAGAAGCCCAGTGATCGGCA
GCGAAGTGTTCCTGCCTAATAGCAATCACGTGGCCAGCGGCGCAG
GAGAAGCCGAGGAGAGAGTGGTCGTGATCAGCAGCAGCGAGGATA
GCGACGCCGAGAATAGCAGCAGCAGAGAGCTGGACGACTCTAGCA
GCGAGTCTAGCGACCTGCAGCTGGAAGGACCTTCCACACTGAGGG
TGCTGGACGAGAACCTGGCAGATCCTCAGGCCGAGGATAGACCTC
TGGTGTTCTTCGACCTGAAGATCGACAACGAGACCCAGAAGATCT
CTCAGCTGGCCGCAGTGAACCGGGAGAGCAAGTTCAGGGTCGTGA
TCCAGCCCGAGGCCTTCTTCAGCATCTACAGCAAGGCCGTGTCTC
TGGAAGTCGGACTGCAGCACTTCCTGAGCTTCCTGAGCAGCATGC
GGAGGCCCATCCTGGCTTGCTACAAGCTCTGGGGCCCAGGCCTGC
CTAATTTCTTCAGAGCCCTCGAGGACATCAACCGCCTCTGGGAGT
TCCAGGAGGCCATTAGCGGATTTCTGGCCGCTCTGCCCCTGATCA
GAGAGAGAGTGCCAGGAGCCAGCAGCTTCAAGCTGAAGAACCTGG
CCCAGACCTACCTGGCTCGGAACATGAGCGAGAGAAGCGCTATGG
CAGCCGTGCTGGCTATGAGAGACCTGTGCAGGCTGCTGGAGGTGT
CACCAGGACCTCAGCTGGCTCAGCACGTGTATCCCTTCAGCAGCC
TCCAGTGTTTCGCCTCTCTGCAGCCTCTGGTGCAGGCAGCAGTGC
TGCCTAGAGCAGAAGCTAGACTGCTGGCCCTGCACAACGTGTCCT
TCATGGAGCTGCTGAGCGCCCATAGAAGAGACAGACAGGGAGGCC
TGAAGAAGTACAGCCGCTACCTGAGCCTGCAGACAACAACACTGC
CTCCAGCCCAGCCAGCTTTCAACCTGCAGGCTCTGGGCACCTACT
TCGAAGGACTGCTGGAAGGACCAGCTCTGGCTAGAGCAGAGGGAG
TGTCTACACCTCTGGCCGGAAGAGGACTGGCAGAAAGAGCCAGCC
AGCAGTCTCACCACCACCATCACCACCACCACTGATGGATCC.

A bolded part represents a restriction site, and the following restriction sites include 8 His tags.

In the present disclosure, the nucleotide sequence shown in SEQ ID NO: 1 is used to construct a lentiviral recombinant expression vector; the nucleotide sequence shown in SEQ ID NO: 15 is used to construct a mammalian recombinant expression vector. The nucleotide sequence shown in SEQ ID NO: 15 is a codon-optimized nucleotide sequence.

In the present invention, the PML-1 protein has two bands, one main band and one secondary band. When a gene encoding PML-1 protein is expressed, two transcripts are produced; a complete PML-1 gene transcript can express a functional PML-1 protein body, with a molecular weight of approximately 110 KD; another transcript, a copy produced during the editing of PML-1, is located at a C-terminus outside an RBCC domain of the PML-1 gene, with an expressed molecular weight of approximately 45 KD.

The present disclosure further provides use of a PML-1 protein and/or a product expressing the PML-1 protein in preparation of a drug for inhibiting a cytokine storm. In the present disclosure, the PML-1 protein and/or product expressing the PML-1 protein can inhibit the synthesis and expression of cytokines by directly inhibiting a signaling pathway of TLRs, thereby inhibiting the cytokine storm.

The present disclosure further provides use of a PML-1 protein and/or a product expressing the PML-1 protein in preparation of an anti-inflammatory drug. The anti-inflammatory drug preferably includes a drug that has a therapeutic effect on inflammations caused by the cytokine storm or inflammatory storm.

The present disclosure provides use of a PML-1 protein and/or a product expressing the PML-1 protein in preparation of a drug for treating a disease with a cytokine storm as a main pathological feature. In the present disclosure, the disease is preferably one or more selected from the group consisting of pneumonia, an autoimmune disease, a tumor, AIDS, and a neurodegenerative disease; the pneumonia preferably includes acute pneumonia; the acute pneumonia is preferably one or more selected from the group consisting of acute lung injury pneumonia, and acute pneumonia caused by the COVID-19 and influenza virus with cytokine storm and inflammatory storm as main pathological features, more preferably the acute lung injury pneumonia. The autoimmune disease preferably includes systemic lupus erythematosus and/or rheumatoid arthritis. The neurodegenerative disease is preferably one or more selected from the group consisting of Alzheimer's disease, Parkinson's disease, multiple sclerosis, and Huntington's disease.

The present disclosure further provides use of a PML-1 protein and/or a product expressing the PML-1 protein in preparation of a drug for regulating an inflammatory signaling pathway. In the present disclosure, the PMIL-1 protein and/or product expressing the PMIL-1 protein can preferably regulate the expression of one or more of TAB1, TAK1/p-TAK1, p-APi, and p-P65 proteins in the inflammatory signaling pathway, more preferably simultaneously regulate the expression of TAB1, TAK1/p-TAK1, p-APi, and p-P65 proteins in the inflammatory signaling pathway. The PML-1 protein and/or product expressing the PML-1 protein preferably regulate the expression of TAB1, TAK1/p-TAK1, p-APi, and p-P65 proteins in the inflammatory signaling pathway, thereby inhibiting the cytokine storm caused by inflammatory cells.

The present disclosure further provides use of a PML-1 protein and/or a product expressing the PMIL-1 protein in preparation of a drug for a regulating inflammatory cytokine. In the present disclosure, the inflammatory cytokine is preferably one or more selected from the group consisting of TNF-α, IL-1β, IL-6, IL-8, MIP-1α, and MCP-1, more preferably the TNF-α, IL-1β, IL-6, IL-8, MIP-1α, and MCP-1. The PML-1 protein and/or product expressing the PMIL-1 protein preferably reduce the expression levels of cytokines such as TNF-α, IL-1β, IL-6, IL-8, MIP-1α, and MCP-1 to inhibit the cytokine storm caused by inflammatory cells.

In the present disclosure, the product expressing the PMIL-1 protein preferably includes a recombinant vector; the recombinant vector preferably includes, but is not limited to, a lentiviral recombinant vector, a mammalian recombinant vector, an adenovirus recombinant vector, or an adeno-associated virus recombinant vector. There are no special restrictions on construction methods of the lentiviral recombinant vector, mammalian recombinant vector, adenovirus recombinant vector, and adeno-associated virus recombinant vector, as long as the expression of PML-1 protein can be achieved using conventional technical means in the field.

The present disclosure further provides a recombinant vector expressing a PML-1 protein, including a base vector and a nucleotide sequence encoding the PML-1 protein; where
the base vector is selected from the group consisting of a lentiviral base vector and a mammalian base vector.

In the present disclosure, a construction method of the lentiviral recombinant vector preferably includes the following steps:
cloning a gene encoding the PML-1 protein into a lentiviral base vector to obtain the lentiviral recombinant vector. There is no special restriction on the lentiviral base vector, as long as it can achieve efficient expression of the PML-1 gene. The lentiviral base vector preferably uses EF1A or EFS as a promoter, more preferably uses the EF1A as the promoter. The EF1A as the promoter can further improve an overexpression effect.

In the present disclosure, the lentiviral base vector preferably uses an SC444-1 vector as a backbone vector to construct the lentiviral base vector using the EF1A as the promoter. Preferably, the EF1A promoter is cloned into the SC444-1 vector to obtain the lentivirus base vector. A restriction enzyme used to clone the EF1A promoter into the SC444-1 vector preferably includes PmlI/BamHI restriction endonucleases. The EFS promoter on the SC444-1 backbone vector is deleted using the PmlI/BamHI restriction endonucleases, and the EF1A promoter is further cloned into the backbone vector to obtain the lentivirus base vector. A restriction enzyme used to clone the PML-1 gene into the lentiviral base vector preferably includes AscI/BamHI restriction endonucleases. There are no special restrictions on reaction conditions during the preparation of lentiviral recombinant vector, and any conventional reaction conditions in this field can be adopted.

In the present disclosure, a construction method of the mammalian recombinant vector preferably includes:
cloning the PML-1 gene into a mammalian base vector to obtain the mammalian recombinant vector.

There is no special restriction on the mammalian base vector, as long as it can achieve efficient expression of the PML-1 gene, and pcDNA3.4 is preferably used. The PML-1 gene is preferably cloned into the pcDNA3.4 plasmid of the mammalian base vector via BamHI/EcoRI restriction endonucleases. There are no special restrictions on the reaction conditions during the construction, and any conventional reaction conditions in this field can be used.

The present disclosure further provides a preparation method of a PML-1 protein and/or a product expressing the PML-1 protein, including expressing the recombinant vector.

In the present disclosure, the product expressing the PML-1 protein is preferably prepared using the lentiviral recombinant vector. The PML-1 protein is preferably prepared using the mammalian recombinant vector.

The present disclosure provides a preparation method of the product expressing the PML-1 protein, including expressing the recombinant vector; where the recombinant vector preferably includes a lentiviral recombinant vector.

In the present disclosure, the lentiviral recombinant vector is preferably co-expressed with a helper plasmid; the helper plasmid preferably includes pLV/helper-SL3, pLV/helper-SL4, and pLV/helper-SL5, abbreviated as SL3, SL4, and SL5.

In the present disclosure, the lentiviral recombinant vector and the helper plasmid are preferably co-transfected into 293T cells for incubation to achieve co-expression of the lentiviral recombinant vector and the helper plasmid. The incubation is conducted at preferably 37° C. for preferably 48 h under preferably 5% CO₂. After the incubation is completed, a cell supernatant is preferably collected by centrifugation.

In the present disclosure, the cell supernatant is preferably filtered by a filter membrane with a pore size of preferably 0.45 m. Cells and cell debris are removed by filtration.

In the present disclosure, a filtered supernatant is preferably mixed with a 40% PEG solution. A mixed solution is allowed to stand for preferably for 3 h to 6 h and preferably on ice.

After the standing is completed, a supernatant is preferably removed by centrifugation, and a precipitate is taken and resuspended to obtain a resuspended lentivirus sample. The resuspension is preferably conducted using a PBS solution. After the resuspension is completed, the resuspended lentivirus sample is preferably transferred to an ultracentrifuge tube for ultracentrifugation at preferably 100,000×g for preferably 90 min. After ultracentrifugation is completed, a supernatant is preferably removed and a precipitate is taken to obtain a lentivirus. The lentivirus is resuspended in a virus storage solution to obtain a lentivirus for transfection. The lentivirus for transfection can preferably be stored at −80° C. The lentivirus prepared by the preparation method has a titer of preferably 4.8×10⁷ TU/mL; the titer of the lentivirus is preferably detected by quantitative PCR (qPCR).

In the present disclosure, the lentivirus for transfection is preferably transfected into cells. The cells preferably include M1 inflammatory macrophages. The M1 inflammatory macrophages are preferably induced by PMA. There is no special limitation on an induction method, and any conventional method in the art can be used.

In the present disclosure, the M1 inflammatory macrophages are preferably M1 inflammatory macrophages in a logarithmic growth phase. Preferably, the M1 inflammatory macrophages are cultured overnight in a 37° C., 5% CO₂ incubator, the old medium in the culture vessel is sucked off; the cells are washed twice with PBS, and ½ volume of fresh medium is added, and then the lentivirus for transfection is added to allow the transfection. The lentivirus for transfection is added at preferably 100 μL. The transfection is conducted for preferably 4 h; the virus liquid and medium are preferably mixed evenly during the transfection. After the transfection is completed, the culture is preferably continued by filling up the remaining ½ volume of medium; the culture is conducted at preferably 37° C. under preferably 5% CO₂ for preferably 48 h. The stably transfected cells are preferably cultured until they reach the logarithmic growth phase, and then the cells are digested and collected.

In the present disclosure, the lentivirus is transfected into cells to express PML-1 protein, thereby inhibiting cytokine storm, regulating inflammatory signaling pathway, and regulating inflammatory cytokine.

The present disclosure provides a preparation method of the PML-1 protein, including expressing the recombinant vector; where the recombinant vector preferably includes a mammalian recombinant vector.

In the present disclosure, a process of expressing the mammalian recombinant vector preferably includes:

transfecting the mammalian recombinant vector into cells, culturing transfected cells, and subjecting collected cells to lysis and purification to obtain the PML-1 protein.

In the present disclosure, before the mammalian recombinant vector is transfected into cells, the mammalian recombinant vector is preferably subjected to bacteriological examination and enzyme digestion identification to determine a reliable overexpression vector. Preferably, the mammalian recombinant vector is transferred to a DH5a cloning strain, a recombinant strain is screened and cultured, and then the mammalian recombinant vector is obtained through a plasmid maximal extraction kit. The DH5a clonal strain preferably includes DH5a competent cells. The transformation preferably includes adding 80 ng to 100 ng of the mammalian recombinant vector into the DH5a competent cells to allow transformation to obtain the recombinant strain.

In the present disclosure, the recombinant strain is preferably cultured in an LB liquid medium at preferably 37° C. for preferably 30 min under preferably 200 rpm. Preferably, a portion of the suspension is taken for screening culture; the screening culture preferably includes culturing the portion of the suspension on an ampicillin-resistant plate at 37° C. overnight until a single colony is obtained. The single colony is preferably selected and cultured in LB liquid medium with a volume of preferably 2 mL to 5 mL at preferably 37° C. for preferably 8 h under preferably 200 rpm. The cultured cells are inoculated into LB liquid medium with a volume of preferably 200 mL at a volume ratio of preferably 1/500 at preferably 37° C. for preferably 16 h under preferably 200 rpm. Preferably, a cultured bacterial solution is collected and centrifuged, and a precipitate is collected to obtain a recombinant strain including the mammalian recombinant vector. Preferably, the mammalian recombinant vector is extracted using a plasmid extraction kit to obtain the mammalian recombinant vector for transfection.

In the present disclosure, the mammalian recombinant vector is transfected into cells; the cells preferably include HEK293 cells.

In the present disclosure, one day before transfection, the HEK293 cells are preferably cultured to control a cell density at (1−1.5)×10⁶ cells/mL; the culture is conducted at preferably 37° C. with preferably 5% CO₂ under preferably 110 rpm. After the culture is completed, the cells for transfection are preferably obtained.

In the present disclosure, the mammalian recombinant vector for transfection is added to the cells for transfection to allow the transfection. The transfected cells are cultured for preferably 4 d to 6 d, more preferably 5 d at preferably 37° C. under preferably 110 rpm with preferably 5% CO₂. After the culture is completed, the cells are preferably collected for lysis and purification to obtain the PML-1 protein. The lysis is preferably conducted using 50 mM Tris, 300 mM NaCl, 8 M Urea, 20 mM Imidazole, and pH=8.0 buffer. After the lysis is completed, purification is conducted, preferably including Ni-IDA affinity chromatography purification and dialysis bag purification. Preferably, the PML-1 protein is obtained after the purification.

The present disclosure further provides a PML-1 protein and/or a product expressing the PML-1 protein prepared by the preparation method.

In the present disclosure, the PML-1 protein and/or the product expressing the PML-1 protein has the effect of inhibiting cytokine storm and can be applied to the treatment of inflammations caused by cytokine storm; the protein or product can be used as a drug to treat diseases with cytokine storm as a main pathological feature; the protein or product can also regulate inflammatory signaling pathway and regulate inflammatory cytokine.

In order to further illustrate the present disclosure, the technical solutions provided by the present disclosure are described in detail below in connection with accompanying drawings and examples, but these examples should not be understood as limiting the claimed scope of the present disclosure.

In the present disclosure, the PML-1 recombinant protein and the PML-1 protein are described as a same substance. The following examples take the mammalian recombinant vector and lentiviral recombinant vector as examples to conduct experiments, so as to illustrate the effects of expressing PML-1 protein. The corresponding effects of the PML-1 protein expressed by adenovirus recombinant vector or adeno-associated virus recombinant vector or product expressing the PML-1 protein are also explained, that is, the effects of Examples 4, 5, and 6 in Implementation Example I and the effects of Examples 4, 5, and 6 in Implementation Example II.

### Implementation Example I

Example 1 Construction of PML-1 overexpression vector (lentiviral recombinant vector)

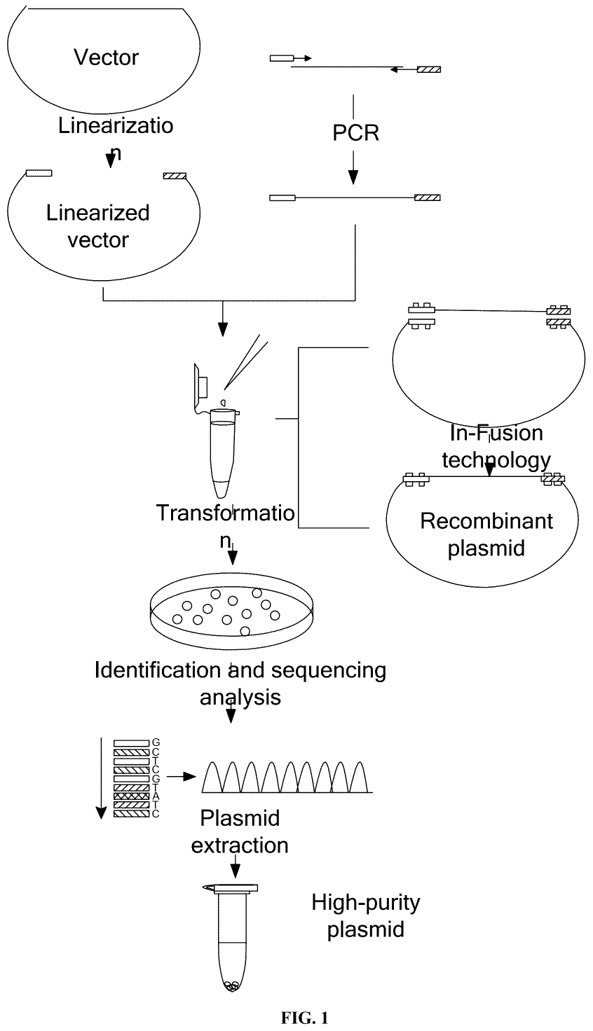

A lentiviral overexpression vector expressing a human PML-1 gene under the control of an EFS promoter was created. Generally speaking: a sequence of a target gene was synthesized, a backbone vector was digested with a restriction endonuclease to obtain a linearized vector, which was ligated to the target gene. The overexpression vector was transformed, screened, and identified using conventional methods, and then used for subsequent virus packaging after plasmid amplification and extraction. The experimental process was shown in FIG. 1, specifically as follows:
1. Whole-gene synthesis of target gene

The PML-1 gene had a CDS sequence [NM_033238.3] shown in SEQ ID NO: 1, and the whole-gene synthesis of the target gene of the PML-1 gene was conducted based on the CDS sequence.
2. An overexpression vector was constructed using an SC444-1 vector as a backbone (the subsequent overexpression project was completed on the basis of LV-EFS>-PGK>EGFP/T2A/Puro).
(1) An EFS promoter on the SC444-1 vector was replaced with an EF1A promoter having a

5′-GGCTCCGGTGCCCGTCAGTGGGCAGAGCGCACATCGCCCACA
GTCCCCGAGAAGTTGGGGGGAGGGGTCGGCAATTGAACCGGTGCC
TAGAGAAGGTGGCGCGGGGTAAACTGGGAAAGTGATGTCGTGTAC
TGGCTCCGCCTTTTTCCCGAGGGTGGGGGAGAACCGTATATAAGT
GCAGTAGTCGCCGTGAACGTTCTTTTTCGCAACGGGTTTGCCGCC
AGAACACAGGTAAGTGCCGTGTGTGGTTCCCGCGGGCCTGGCCTC
TTTACGGGTTATGGCCCTTGCGTGCCTTGAATTACTTCCACCTGG
CTGCAGTACGTGATTCTTGATCCCGAGCTTCGGGTTGGAAGTGGG
TGGGAGAGTTCGAGGCCTTGCGCTTAAGGAGCCCCTTCGCCTCGT
GCTTGAGTTGAGGCCTGGCCTGGGCGCTGGGGCCGCCGCGTGCGA
ATCTGGTGGCACCTTCGCGCCTGTCTCGCTGCTTTCGATAAGTCT
CTAGCCATTTAAAATTTTTGATGACCTGCTGCGACGCTTTTTTTC
TGGCAAGATAGTCTTGTAAATGCGGGCCAAGATCTGCACACTGGT
ATTTCGGTTTTTGGGGCCGCGGGCGGCGACGGGGCCCGTGCGTCC
CAGCGCACATGTTCGGCGAGGCGGGGCCTGCGAGCGCGGCCACCG
AGAATCGGACGGGGGTAGTCTCAAGCTGGCCGGCCTGCTCTGGTG
CCTGGTCTCGCGCCGCCGTGTATCGCCCCGCCCTGGGCGGCAAGG
CTGGCCCGGTCGGCACCAGTTGCGTGAGCGGAAAGATGGCCGCTT
CCCGGCCCTGCTGCAGGGAGCTCAAAATGGAGGACGCGGCGCTCG
GGAGAGCGGGCGGGTGAGTCACCCACACAAAGGAAAAGGGCCTTT
CCGTCCTCAGCCGTCGCTTCATGTGACTCCACGGAGTACCGGGCG
CCGTCCAGGCACCTCGATTAGTTCTCGAGCTTTTGGAGTACGTCG
TCTTTAGGTTGGGGGGAGGGGTTTTATGCGATGGAGTTTCCCCAC
ACTGAGTGGGTGGAGACTGAAGTTAGGCCAGCTTGGCACTTGATG
TAATTCTCCTTGGAATTTGCCCTTTTTGAGTTTGGATCTTGGTTC
ATTCTCAAGCCTCAGACAGTGGTTCAAAGTTTTTTTCTTCCATTT
CAGGTGTCGTGA-3′.

The SC444-1 vector was digested with PmlI/BamHI restriction endonucleases, where an enzyme digestion system included: 17.5 μL of Cutsmart, 600 ng to 800 ng of plasmid (SC444-1 vector), 0.5 μL of PmII, and 0.5 μL of BamHI; the enzyme digestion was conducted at 37° C. for 1 h.

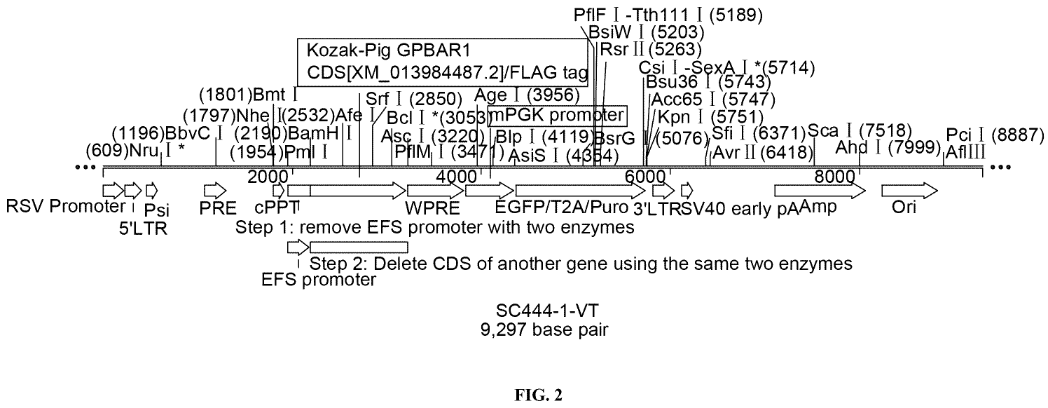

After the enzyme digestion was completed, a gel was spotted and recovered, and a band was recovered using a gel recovery kit (NEB Monarch DNA) to obtain a linearized vector. The EF1A promoter sequence and the linearized SC444-1 vector were ligated using a C115 Infusion ligase system (C115 enzyme from Vazyme Biotech), where the ligase systems included: 100 ng of linearized vector, 50 ng of target fragment (EF1A promoter sequence), 5 μL of ligase, and making up to 10 μL with sterile water. The ligation was conducted at 50° C. and a hot lid temperature of 105° C. for 15 min. The backbone vector containing the EF1A promoter was obtained through the reaction.
(2) The backbone vector containing the EF1A promoter was digested with AscI/BamHI restriction endonucleases and gel-recovered according to the conditions the same as those in step (1). The target gene of PML-1 gene and the recovered backbone vector containing the EF1A promoter were ligated using the C115 Infusion ligase system according to the ligase system and ligation conditions the same as those in step (1). The obtained recombinant plasmid could be called SC493-1 plasmid, which was the overexpression vector of PML-1 gene. A structural schematic diagram of the complete information of the plasmid was shown in FIG. 2.
3. After the ligation was completed, a plate (LB solid medium) with the corresponding resistance (Amp concentration of 100 g/mL) was dried in a 37° C. incubator upside down, while 500 μL of antibiotic-free medium (LB culture medium) was preheated. The competent cells (DH5a competent cells.) were taken out from −80° C. The competent cells were placed on ice for 15 min to 20 min until melt, half of the ligation solution was added into the competent cells, flicked 5 times with fingers, and treated in ice bath for 30 min; the competent cells were quickly transferred to a preheated 42° C. water bath and heat-shocked for 1 min (heat shock for 45 s to 90 s with strict time control); when the heat shock was completed, the cells were immediately transferred to ice and placed for 2 min, then transferred to 500 μL of a preheated medium and cultured on a 37° C. shaker for 1 h; a bacterial solution was centrifuged at 4,000 rpm for 2 min to remove a supernatant (400 μL of the supernatant was discarded); about 20 glass beads were poured on the plate, the bacterial solution were mixed well by pipetting and then spread onto the plate with corresponding resistance (LB solid medium with Amp concentration of 100 g/mL), and cultured in a 37° C. incubator overnight.
4. Bacterial examination and extraction of recombinant plasmid (PML-1 gene overexpression vector)

The ultraviolet of an ultra-clean bench was turned on; after 20 min, the ultraviolet was turned off while the third wind speed was started, the ultra-clean bench was wiped with 75% ethanol to start the bacterial examination. On the ultra-clean bench, 25 μL of sterilized water was added to the bottom of a 96-well PCR plate, it was checked if there was air at the bottom against the light; the monoclonal plaques on the overnight culture plate were gently dipped with a sterilized 10 L white pipette tip, transferred into the corresponding 96-well reaction plate, mixed well by pipetting with a 10 μL row pipet 2 to 3 times, and 4 μL of samples were pipetted row by row to a new 96-well plate as a template to allow PCR reaction for bacterial examination. 50 μL of antibiotic-free broth medium was added to the original 96-well plate, covered by the sterilized 96-well plate and placed in a 30° C. incubator.

A PCR reaction system for bacterial examination included: 12.5 μL of 2× Taq enzyme mixture (P222 purchased from Vazyme Biotech), 4 μL of single bacterial colony suspension, 0.75 L each of upstream and downstream primers, and making up to 25 μL with sterile water. A PCR program included: initial denaturation at 95° C. for 3 min; 23 cycles of denaturation at 95° C. for 15 s, annealing at 60° C. for 15 s, and extension at 72° C. for 15 s/kb; final extension at 72° C. for 5 min; and storage at 25° C. The primers for bacteriological examination were shown in Table 1.

TABLE 1
Primer information for bacteriological
examination

| | Primer | Primer | |
|---|---|---|---|
| | name | ID | 5′-3′ |
| Examination | SC493-1-P1 | SEQ ID | actagtgatt |
| of EF1A | SC493-1-P2 | NO: 4 | atcggattga |
| promoter | | SEQ ID | GAAGCTCGGG |
| | | NO: 5 | ATCAAGAATC |
| | SC493-1-P3 | SEQ ID | cttcatgtga |
| | | NO: 6 | ctccacggag |
| | SC493-1-P4 | SEQ ID | CAGGTTGGCA |
| | | NO: 7 | GCAACGATGA |
| Examination | SC493-1-P3 | SEQ ID | cttcatgtga |
| of PML-1 | | NO: 6 | ctccacggag |
| | SC493-1-P5 | SEQ ID | AGGCATCCTG |
| | | NO: 8 | AGCACAGCGT |
| | | | GT |
| | SC493-1-P6 | SEQ ID | aactcaagaa |
| | | NO: 9 | cctggcccag |
| | SC493-1-P7 | SEQ ID | CGTCAGCAAA |
| | | NO: 10 | CACAGTGCAC |

Notes:
P1/P2 were used in combination; P3/P4 were used in combination; P3/P5 were used in combination; P6/P7 were used in combination.

After the PCR was completed, electrophoresis was conducted with a TBE detection gel to check whether a band size was consistent with a theoretical band size. The consistent recombinant strain was then cultured in 3.5 mL of 100 g/mL Amp-resistant broth in a 48-deep well plate and placed on a 37° C. shaker for overnight culture.

A plasmid of the positive recombinant strain was extracted to obtain a recombinant plasmid, which was then sequenced and identified by enzyme digestion to determine the accurate recombinant plasmid.

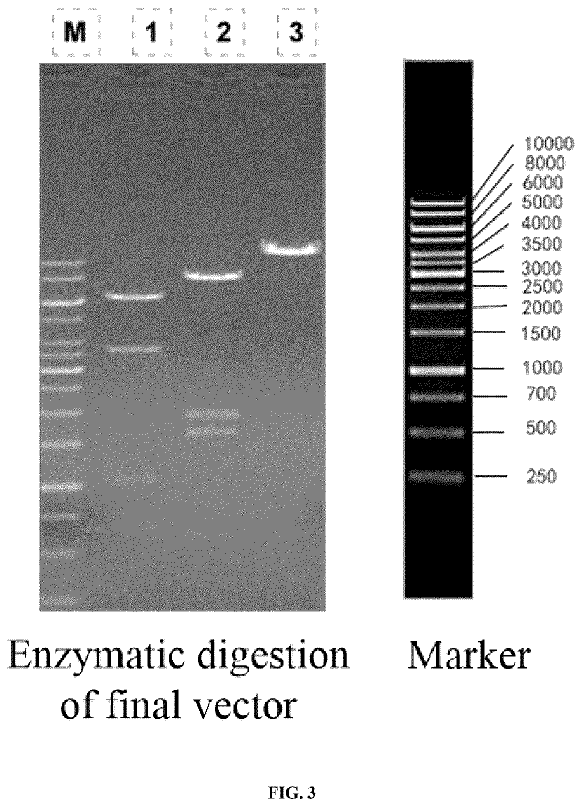

Based on the existing endonucleases, NTI/SnapGene was used to search for available restriction enzyme digestion plans to identify whether the plasmid was correct, where enzyme digestion system and conditions were the same as those in step (1), and the enzymes for restriction digestion were Afl1I/MfeI and AhdI/XmnI, and AscI. The results of enzyme digestion identification of the recombinant plasmid were shown in FIG. 3. In FIG. 3, 1 corresponded to the enzyme digestion product of the Afl1I/MfeI restriction endonucleases; 2 corresponded to the enzyme digestion product of the AhdI/XmnI restriction endonucleases; 3 corresponded to the enzyme digestion product of the AscI restriction endonuclease; and M represented marker.

According to the enzyme digestion identification results, the Afl1I/MfeI digested the recombinant plasmid into fragments of 6.2 kb, 3.7 kb, 1.1 kb, 0.7 kb, and 0.3 kb; the AhdI/XmnI digested the recombinant plasmid into fragments of 7.7 kb, 1.9 kb, 1.6 kb, and 0.6 kb; the AscI digested the recombinant plasmid into a fragment of 11.9 kb. The recombinant plasmid was the overexpression vector of the PML-1 gene, also called a lentiviral plasmid.

The overexpression vector of the PML-1 gene had a nucleotide sequence shown in SEQ ID NO: 2, specifically:

AATGTAGTCTTATGCAATACTCTTGTAGTCTTGCAACATGGTAACGATGAG
TTAGCAACATGCCTTACAAGGAGAGAAAAAGCACCGTGCATGCCGATTGG
TGGAAGTAAGGTGGTACGATCGTGCCTTATTAGGAAGGCAACAGACGGGT
CTGACATGGATTGGACGAACCACTGAATTGCCGCATTGCAGAGATATTGT
ATTTAAGTGCCTAGCTCGATACATAAACGGGTCTCTCTGGTTAGACCAGA
TCTGAGCCTGGGAGCTCTCTGGCTAACTAGGGAACCCACTGCTTAAGCCT
CAATAAAGCTTGCCTTGAGTGCTTCAAGTAGTGTGTGCCCGTCTGTTGTG
TGACTCTGGTAACTAGAGATCCCTCAGACCCTTTTAGTCAGTGTGGAAAA
TCTCTAGCAGTGGCGCCCGAACAGGGACTTGAAAGCGAAAGGGAAACCAG
AGGAGCTCTCTCGACGCAGGACTCGGCTTGCTGAAGCGCGCACGGCAAGA
GGCGAGGGGCGGCGACTGGTGAGTACGCCAAAAATTTTGACTAGCGGAGG
CTAGAAGGAGAGAGATGGGTGCGAGAGCGTCAGTATTAAGCGGGGGAGAA
TTAGATCGCGATGGGAAAAAATTCGGTTAAGGCCAGGGGGAAAGAAAAAA
TATAAATTAAAACATATAGTATGGGCAAGCAGGGAGCTAGAACGATTCGC
AGTTAATCCTGGCCTGTTAGAAACATCAGAAGGCTGTAGACAAATACTGG
GACAGCTACAACCATCCCTTCAGACAGGATCAGAAGAACTTAGATCATTA
TATAATACAGTAGCAACCCTCTATTGTGTGCATCAAAGGATAGAGATAAA
AGACACCAAGGAAGCTTTAGACAAGATAGAGGAAGAGCAAAACAAAAGTA
AGACCACCGCACAGCAAGCGGCCGCTGATCTTCAGACCTGGAGGAGGAGA
TATGAGGGACAATTGGAGAAGTGAATTATATAAATATAAAGTAGTAAAAA
TTGAACCATTAGGAGTAGCACCCACCAAGGCAAAGAGAAGAGTGGTGCAG
AGAGAAAAAAGAGCAGTGGGAATAGGAGCTTTGTTCCTTGGGTTCTTGGG
AGCAGCAGGAAGCACTATGGGCGCAGCGTCAATGACGCTGACGGTACAGG
CCAGACAATTATTGTCTGGTATAGTGCAGCAGCAGAACAATTTGCTGAGG
GCTATTGAGGCGCAACAGCATCTGTTGCAACTCACAGTCTGGGGCATCAA
GCAGCTCCAGGCAAGAATCCTGGCTGTGGAAAGATACCTAAAGGATCAAC
AGCTCCTGGGGATTTGGGGTTGCTCTGGAAAACTCATTTGCACCACTGCT
GTGCCTTGGAATGCTAGTTGGAGTAATAAATCTCTGGAACAGATTTGGAA
TCACACGACCTGGATGGAGTGGGACAGAGAAATTAACAATTACACAAGCT
TAATACACTCCTTAATTGAAGAATCGCAAAACCAGCAAGAAAAGAATGAA
CAAGAATTATTGGAATTAGATAAATGGGCAAGTTTGTGGAATTGGTTTAA
CATAACAAATTGGCTGTGGTATATAAAATTATTCATAATGATAGTAGGAG
GCTTGGTAGGTTTAAGAATAGTTTTTGCTGTACTTTCTATAGTGAATAGA
GTTAGGCAGGGATATTCACCATTATCGTTTCAGACCCACCTCCCAACCCC
GAGGGGACCCGACAGGCCCGAAGGAATAGAAGAAGAAGGTGGAGAGAGAG
ACAGAGACAGATCCATTCGATTAGTGAACGGATCTCGACGGTATCGCTAG
CTTTTAAAAGAAAAGGGGGGATTGGGGGGTACAGTGCAGGGGAAAGAATA
GTAGACATAATAGCAACAGACATACAAACTAAAGAATTACAAAAACAAAT
TACAAAAATTCAAAATTTTACTAGTGATTATCGGATTGATAATCGAATTC
CACGTGGGCTCCGGTGCCCGTCAGTGGGCAGAGCGCACATCGCCCACAGT
CCCCGAGAAGTTGGGGGGAGGGGTCGGCAATTGAACCGGTGCCTAGAGAA
GGTGGCGCGGGGTAAACTGGGAAAGTGATGTCGTGTACTGGCTCCGCCTT
TTTCCCGAGGGTGGGGGAGAACCGTATATAAGTGCAGTAGTCGCCGTGAA
CGTTCTTTTTCGCAACGGGTTTGCCGCCAGAACACAGGTAAGTGCCGTGT
GTGGTTCCCGCGGGCCTGGCCTCTTTACGGGTTATGGCCCTTGCGTGCCT
TGAATTACTTCCACCTGGCTGCAGTACGTGATTCTTGATCCCGAGCTTCG
GGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTG
CGAATCTGGTGGCACCTTCGCGCCTGTCTCGCTGCTTTCGATAAGTCTCT
AGCCATTTAAAATTTTTGATGACCTGCTGCGACGCTTTTTTTCTGGCAAG
ATAGTCTTGTAAATGCGGGCCAAGATCTGCACACTGGTATTTCGGTTTTT
GGGGCCGCGGGCGGCGACGGGGCCCGTGCGTCCCAGCGCACATGTTCGGC
GAGGCGGGGCCTGCGAGCGCGGCCACCGAGAATCGGACGGGGGTAGTCTC
AAGCTGGCCGGCCTGCTCTGGTGCCTGGTCTCGCGCCGCCGTGTATCGCC
CCGCCCTGGGCGGCAAGGCTGGCCCGGTCGGCACCAGTTGCGTGAGCGGA
AAGATGGCCGCTTCCCGGCCCTGCTGCAGGGAGCTCAAAATGGAGGACGC
GGCGCTCGGGAGAGCGGGCGGGTGAGTCACCCACACAAAGGAAAAGGGCC
TTTCCGTCCTCAGCCGTCGCTTCATGTGACTCCACGGAGTACCGGGCGCC
GTCCAGGCACCTCGATTAGTTCTCGAGCTTTTGGAGTACGTCGTCTTTAG
GTTGGGGGGAGGGGTTTTATGCGATGGAGTTTCCCCACACTGAGTGGGTG
GAGACTGAAGTTAGGCCAGCTTGGCACTTGATGTAATTCTCCTTGGAATT
TGCCCTTTTTGAGTTTGGATCTTGGTTCATTCTCAAGCCTCAGACAGTGG
TTCAAAGTTTTTTTCTTCCATTTCAGGTGTCGTGAGGATCCGCCGCCACC
ATGGAGCCTGCACCCGCCCGATCTCCGAGGCCCCAGCAGGACCCCGCCCG
GCCCCAGGAGCCCACCATGCCTCCCCCCGAGACCCCCTCTGAAGGCCGCC
AGCCCAGCCCCAGCCCCAGCCCTACAGAGCGAGCCCCCGCTTCGGAGGAG
GAGTTCCAGTTTCTGCGCTGCCAGCAATGCCAGGCGGAAGCCAAGTGCCC
GAAGCTGCTGCCTTGTCTGCACACGCTGTGCTCAGGATGCCTGGAGGCGT
CGGGCATGCAGTGCCCCATCTGCCAGGCGCCCTGGCCCCTAGGTGCAGAC
ACACCCGCCCTGGATAACGTCTTTTTCGAGAGTCTGCAGCGGCGCCTGTC
GGTGTACCGGCAGATTGTGGATGCGCAGGCTGTGTGCACCCGCTGCAAAG
AGTCGGCCGACTTCTGGTGCTTTGAGTGCGAGCAGCTCCTCTGCGCCAAG
TGCTTCGAGGCACACCAGTGGTTCCTCAAGCACGAGGCCCGGCCCCTAGC
AGAGCTGCGCAACCAGTCGGTGCGTGAGTTCCTGGACGGCACCCGCAAGA
CCAACAACATCTTCTGCTCCAACCCCAACCACCGCACCCCTACGCTGACC
AGCATCTACTGCCGAGGATGTTCCAAGCCGCTGTGCTGCTCGTGCGCGCT
CCTTGACAGCAGCCACAGTGAGCTCAAGTGCGACATCAGCGCAGAGATCC
AGCAGCGACAGGAGGAGCTGGACGCCATGACGCAGGCGCTGCAGGAGCAG
GATAGTGCCTTTGGCGCGGTTCACGCGCAGATGCACGCGGCCGTCGGCCA
GCTGGGCCGCGCGCGTGCCGAGACCGAGGAGCTGATCCGCGAGCGCGTGC
GCCAGGTGGTAGCTCACGTGCGGGCTCAGGAGCGCGAGCTGCTGGAGGCT
GTGGACGCGCGGTACCAGCGCGACTACGAGGAGATGGCCAGTCGGCTGGG
CCGCCTGGATGCTGTGCTGCAGCGCATCCGCACGGGCAGCGCGCTGGTGC
AGAGGATGAAGTGCTACGCCTCGGACCAGGAGGTGCTGGACATGCACGGT
TTCCTGCGCCAGGCGCTCTGCCGCCTGCGCCAGGAGGAGCCCCAGAGCCT
GCAAGCTGCCGTGCGCACCGATGGCTTCGACGAGTTCAAGGTGCGCCTGC
AGGACCTCAGCTCTTGCATCACCCAGGGGAAAGATGCAGCTGTATCCAAG
AAAGCCAGCCCAGAGGCTGCCAGCACTCCCAGGGACCCTATTGACGTTGA
CCTGCCCGAGGAGGCAGAGAGAGTGAAGGCCCAGGTTCAGGCCCTGGGGC
TGGCTGAAGCCCAGCCTATGGCTGTGGTACAGTCAGTGCCCGGGGCACAC
CCCGTGCCAGTGTACGCCTTCTCCATCAAAGGCCCTTCCTATGGAGAGGA
TGTCTCCAATACAACGACAGCCCAGAAGAGGAAGTGCAGCCAGACCCAGT
GCCCCAGGAAGGTCATCAAGATGGAGTCTGAGGAGGGGAAGGAGGCAAGG
TTGGCTCGGAGCTCCCCGGAGCAGCCCAGGCCCAGCACCTCCAAGGCAGT
CTCACCACCCCACCTGGATGGACCGCCTAGCCCCAGGAGCCCCGTCATAG
GAAGTGAGGTCTTCCTGCCCAACAGCAACCACGTGGCCAGTGGCGCCGGG
GAGGCAGAGGAACGCGTTGTGGTGATCAGCAGCTCGGAAGACTCAGATGC
CGAAAACTCGTCCTCCCGAGAGCTGGATGACAGCAGCAGTGAGTCCAGTG
ACCTCCAGCTGGAAGGCCCCAGCACCCTCAGGGTCCTGGACGAGAACCTT
GCTGACCCCCAAGCAGAAGACAGACCTCTGGTTTTCTTTGACCTCAAGAT
TGACAATGAAACCCAGAAGATTAGCCAGCTGGCTGCGGTGAACCGGGAAA
GCAAGTTCCGCGTGGTCATCCAGCCTGAAGCCTTCTTCAGCATCTACTCC
AAGGCCGTGTCCCTGGAGGTGGGGCTGCAGCACTTCCTCAGCTTTCTGAG
CTCCATGCGCCGCCCTATCTTGGCCTGCTACAAGCTGTGGGGGCCTGGCC
TCCCAAACTTCTTCCGGGCCCTGGAGGACATTAACAGGCTGTGGGAATTC
CAGGAGGCCATCTCGGGCTTCCTGGCTGCCCTGCCTCTCATCCGGGAGCG
TGTGCCCGGGGCCAGCAGCTTCAAACTCAAGAACCTGGCCCAGACCTACC
TGGCGAGAAACATGAGCGAGCGCAGCGCCATGGCTGCCGTGCTGGCCATG
CGTGACCTGTGCCGCCTCCTCGAGGTCTCCCCGGGCCCCCAGCTGGCCCA
GCATGTCTACCCCTTCAGTAGCCTGCAGTGCTTTGCCTCCCTGCAGCCCC
TGGTGCAGGCAGCTGTGCTGCCCCGGGCTGAGGCCCGCCTCCTGGCCCTA
CACAACGTGAGCTTCATGGAGCTGCTGAGTGCACACCGCCGTGACCGGCA
GGGGGGCCTGAAGAAGTACAGCCGCTATCTAAGCCTGCAGACCACCACGT
TGCCCCCTGCCCAGCCTGCTTTCAACCTGCAGGCTCTGGGCACCTACTTT
GAAGGCCTGTTGGAGGGTCCGGCGCTGGCACGGGCAGAAGGAGTCTCCAC
CCCACTTGCTGGCCGTGGCTTGGCAGAGAGGGCCTCCCAGCAGAGCTGAG
GCGCGCCGATAATCAACCTCTGGATTACAAAATTTGTGAAAGATTGACTG
GTATTCTTAACTATGTTGCTCCTTTTACGCTATGTGGATACGCTGCTTTA
ATGCCTTTGTATCATGCTATTGCTTCCCGTATGGCTTTCATTTTCTCCTC
CTTGTATAAATCCTGGTTGCTGTCTCTTTATGAGGAGTTGTGGCCCGTTG
TCAGGCAACGTGGCGTGGTGTGCACTGTGTTTGCTGACGCAACCCCCACT
GGTTGGGGCATTGCCACCACCTGTCAGCTCCTTTCCGGGACTTTCGCTTT
CCCCCTCCCTATTGCCACGGCGGAACTCATCGCCGCCTGCCTTGCCCGCT
GCTGGACAGGGGCTCGGCTGTTGGGCACTGACAATTCCGTGGTGTTGTCG
GGGAAGCTGACGTCCTTTCCATGGCTGCTCGCCTGTGTTGCCACCTGGAT
TCTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCTT
CGCCTTCGCCCTCAGACGAGTCGGATCTCCCTTTGGGCCGCCTCCCCGCA
TCGGGAATTCCCGCGGTTCTTCTACCGGGTAGGGGAGGCGCTTTTCCCAA
GGCAGTCTGGAGCATGCGCTTTAGCAGCCCCGCTGGGCACTTGGCGCTAC
ACAAGTGGCCTCTGGCCTCGCACACATTCCACATCCACCGGTAGGCGCCA
ACCGGCTCCGTTCTTTGGTGGCCCCTTCGCGCCACCTTCTACTCCTCCCC
TAGTCAGGAAGTTCCCCCCCGCCCCGCAGCTCGCGTCGTGCAGGACGTGA
CAAATGGAAGTAGCACGTCTCACTAGTCTCGTGCAGATGGACAGCACCGC
TGAGCAATGGAAGCGGGTAGGCCTTTGGGGCAGCGGCCAATAGCAGCTTT
GCTCCTTCGCTTTCTGGGCTCAGAGGCTGGGAAGGGGTGGGTCCGGGGGC
GGGCTCAGGGGCGGGCTCAGGGGCGGGGGGGGCGCCCGAAGGTCCTCCGG
AGGCCCGGCATTCTGCACGCTTCAAAAGCGCACGTCTGCCGCGCTGTTCT
CCTCTTCCTCATCTCCGGGCCTTTCGACCTGCGATCGCGCCGCCACCATG
GTGAGCAAGGGCGAGGAGCTGTTCACCGGGGTGGTGCCCATCCTGGTCGA
GCTGGACGGCGACGTAAACGGCCACAAGTTCAGCGTGTCCGGCGAGGGCG
AGGGCGATGCCACCTACGGCAAGCTGACCCTGAAGTTCATCTGCACCACC
GGCAAGCTGCCCGTGCCCTGGCCCACCCTCGTGACCACCCTGACCTACGG
CGTGCAGTGCTTCAGCCGCTACCCCGACCACATGAAGCAGCACGACTTCT
TCAAGTCCGCCATGCCCGAAGGCTACGTCCAGGAGCGCACCATCTTCTTC
AAGGACGACGGCAACTACAAGACCCGCGCCGAGGTGAAGTTCGAGGGCGA
CACCCTGGTGAACCGCATCGAGCTGAAGGGCATCGACTTCAAGGAGGACG
GCAACATCCTGGGGCACAAGCTGGAGTACAACTACAACAGCCACAACGTC
TATATCATGGCCGACAAGCAGAAGAACGGCATCAAGGTGAACTTCAAGAT
CCGCCACAACATCGAGGACGGCAGCGTGCAGCTCGCCGACCACTACCAGC
AGAACACCCCCATCGGCGACGGCCCCGTGCTGCTGCCCGACAACCACTAC
CTGAGCACCCAGTCCGCCCTGAGCAAAGACCCCAACGAGAAGCGCGATCA
CATGGTCCTGCTGGAGTTCGTGACCGCCGCCGGGATCACTCTCGGCATGG
ACGAGCTGTACAAGGGCTCCGGAGAGGGCAGGGGAAGTCTTCTAACATGC
GGGGACGTGGAGGAAAATCCCGGCCCCATGACCGAGTACAAGCCCACGGT
GCGCCTCGCCACCCGCGACGACGTCCCCAGGGCCGTACGCACCCTCGCCG
CCGCGTTCGCCGACTACCCCGCCACGCGCCACACCGTCGATCCGGACCGC
CACATCGAGCGGGTCACCGAGCTGCAAGAACTCTTCCTCACGCGCGTCGG
GCTCGACATCGGCAAGGTGTGGGTCGCGGACGACGGCGCCGCGGTGGCGG
TCTGGACCACGCCGGAGAGCGTCGAAGCGGGGGCGGTGTTCGCCGAGATC
GGCCCGCGCATGGCCGAGTTGAGCGGTTCCCGGCTGGCCGCGCAGCAACA
GATGGAAGGCCTCCTGGCGCCGCACCGGCCCAAGGAGCCCGCGTGGTTCC
TGGCCACCGTCGGCGTCTCGCCCGACCACCAGGGCAAGGGTCTGGGCAGC
GCCGTCGTGCTCCCCGGAGTGGAGGCGGCCGAGCGCGCCGGGGTGCCCGC
CTTCCTGGAGACCTCCGCGCCCCGCAACCTCCCCTTCTACGAGCGGCTCG
GCTTCACCGTCACCGCCGACGTCGAGGTGCCCGAAGGACCGCGCACCTGG
TGCATGACCCGCAAGCCCGGTGCCTGAGGTACCTTTAAGACCAATGACTT
ACAAGGCAGCTGTAGATCTTAGCCACTTTTTAAAAGAAAAGGGGGGACTG
GAAGGGCTAATTCACTCCCAACGAAGACAAGATCTGCTTTTTGCTTGTAC
TGGGTCTCTCTGGTTAGACCAGATCTGAGCCTGGGAGCTCTCTGGCTAAC
TAGGGAACCCACTGCTTAAGCCTCAATAAAGCTTGCCTTGAGTGCTTCAA
GTAGTGTGTGCCCGTCTGTTGTGTGACTCTGGTAACTAGAGATCCCTCAG
ACCCTTTTAGTCAGTGTGGAAAATCTCTAGCAGTAGTAGTTCATGTCATC
TTATTATTCAGTATTTATAACTTGCAAAGAAATGAATATCAGAGAGTGAG
AGGAACTTGTTTATTGCAGCTTATAATGGTTACAAATAAAGCAATAGCAT
CACAAATTTCACAAATAAAGCATTTTTTTCACTGCATTCTAGTTGTGGTT
TGTCCAAACTCATCAATGTATCTTATCATGTCTGGCTCTAGCTATCCCGC
CCCTAACTCCGCCCATCCCGCCCCTAACTCCGCCCAGTTCCGCCCATTCT
CCGCCCCATGGCTGACTAATTTTTTTTATTTATGCAGAGGCCGAGGCCGC
CTCGGCCTCTGAGCTATTCCAGAAGTAGTGAGGAGGCTTTTTTGGAGGCC
TAGGGACGTACCCAATTCGCCCTATAGTGAGTCGTATTACGCGCGCTCAC
TGGCCGTCGTTTTACAACGTCGTGACTGGGAAAACCCTGGCGTTACCCAA
CTTAATCGCCTTGCAGCACATCCCCCTTTCGCCAGCTGGCGTAATAGCGA
AGAGGCCCGCACCGATCGCCCTTCCCAACAGTTGCGCAGCCTGAATGGCG
AATGGGACGCGCCCTGTAGCGGCGCATTAAGCGCGGCGGGTGTGGTGGTT
ACGCGCAGCGTGACCGCTACACTTGCCAGCGCCCTAGCGCCCGCTCCTTT
CGCTTTCTTCCCTTCCTTTCTCGCCACGTTCGCCGGCTTTCCCCGTCAAG
CTCTAAATCGGGGGCTCCCTTTAGGGTTCCGATTTAGTGCTTTACGGCAC
CTCGACCCCAAAAAACTTGATTAGGGTGATGGTTCACGTAGTGGGCCATC
GCCCTGATAGACGGTTTTTCGCCCTTTGACGTTGGAGTCCACGTTCTTTA
ATAGTGGACTCTTGTTCCAAACTGGAACAACACTCAACCCTATCTCGGTC
TATTCTTTTGATTTATAAGGGATTTTGCCGATTTCGGCCTATTGGTTAAA
AAATGAGCTGATTTAACAAAAATTTAACGCGAATTTTAACAAAATATTAA
CGCTTACAATTTAGGTGGCACTTTTCGGGGAAATGTGCGCGGAACCCCTA
TTTGTTTATTTTTCTAAATACATTCAAATATGTATCCGCTCATGAGACAA
TAACCCTGATAAATGCTTCAATAATATTGAAAAAGGAAGAGTATGAGTAT
TCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGAT
CAGTTGGGTGCACGAGTGGGTTACATCGAACTGGATCTCAACAGCGGTAA
GATCCTTGAGAGTTTTCGCCCCGAAGAACGTTTTCCAATGATGAGCACTT
TTAAAGTTCTGCTATGTGGCGCGGTATTATCCCGTATTGACGCCGGGCAA
GAGCAACTCGGTCGCCGCATACACTATTCTCAGAATGACTTGGTTGAGTA
CTCACCAGTCACAGAAAAGCATCTTACGGATGGCATGACAGTAAGAGAAT
TATGCAGTGCTGCCATAACCATGAGTGATAACACTGCGGCCAACTTACTT
CTGACAACGATCGGAGGACCGAAGGAGCTAACCGCTTTTTTGCACAACAT
GGGGGATCATGTAACTCGCCTTGATCGTTGGGAACCGGAGCTGAATGAAG
CCATACCAAACGACGAGCGTGACACCACGATGCCTGTAGCAATGGCAACA
ACGTTGCGCAAACTATTAACTGGCGAACTACTTACTCTAGCTTCCCGGCA
ACAATTAATAGACTGGATGGAGGCGGATAAAGTTGCAGGACCACTTCTGC
GCTCGGCCCTTCCGGCTGGCTGGTTTATTGCTGATAAATCTGGAGCCGGT
GAGCGTGGGTCTCGCGGTATCATTGCAGCACTGGGGCCAGATGGTAAGCC
CTCCCGTATCGTAGTTATCTACACGACGGGGAGTCAGGCAACTATGGATG
AACGAAATAGACAGATCGCTGAGATAGGTGCCTCACTGATTAAGCATTGG
TAACTGTCAGACCAAGTTTACTCATATATACTTTAGATTGATTTAAAACT
TCATTTTTAATTTAAAAGGATCTAGGTGAAGATCCTTTTTGATAATCTCA
TGACCAAAATCCCTTAACGTGAGTTTTCGTTCCACTGAGCGTCAGACCCC
GTAGAAAAGATCAAAGGATCTTCTTGAGATCCTTTTTTTCTGCGCGTAAT
CTGCTGCTTGCAAACAAAAAAACCACCGCTACCAGCGGTGGTTTGTTTGC
CGGATCAAGAGCTACCAACTCTTTTTCCGAAGGTAACTGGCTTCAGCAGA
GCGCAGATACCAAATACTGTTCTTCTAGTGTAGCCGTAGTTAGGCCACCA
CTTCAAGAACTCTGTAGCACCGCCTACATACCTCGCTCTGCTAATCCTGT
TACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGG
TTCGTGCACACAGCCCAGCTTGGAGCGAACGACCTACACCGAACTGAGAT
ACCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGAG
GGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTT
TTGCTGGCCTTTTGCTCACATGTTCTTTCCTGCGTTATCCCCTGATTCTG
TGGATAACCGTATTACCGCCTTTGAGTGAGCTGATACCGCTCGCCGCAGC
CGAACGACCGAGCGCAGCGAGTCAGTGAGCGAGGAAGCGGAAGAGCGCCC
AATACGCAAACCGCCTCTCCCCGCGCGTTGGCCGATTCATTAATGCAGCT
GGCACGACAGGTTTCCCGACTGGAAAGCGGGCAGTGAGCGCAACGCAATT
AATGTGAGTTAGCTCACTCATTAGGCACCCCAGGCTTTACACTTTATGCT
TCCGGCTCGTATGTTGTGTGGAATTGTGAGCGGATAACAATTTCACACAG
GAAACAGCTATGACCATGATTACGCCAAGCGCGCAATTAACCCTCACTAA
AGGGAACAAAAGCTGGAGCTGCAAGCTT.

### Example 2 Construction of Lentivirus Expressing PML-1 Protein

1. Lentivirus package

The lentiviral plasmid and helper plasmids (SL3, SL4 and SL5) were co-infected into 293T cells. After incubation for 48 h in a 5% CO₂ and 37° C. incubator, a cell supernatant was collected by centrifugation. The cell supernatant was filtered using a 0.45 m filter to remove cells and cell debris; the supernatant was mixed with 40% PEG solution and placed on ice for 3 h to 6 h; after centrifugation for 30 min, a supernatant was removed, the virus sample was resuspended in PBS, transferred to an ultracentrifuge tube, placed in an ultracentrifuge and centrifuged at 100,000×g (24,500 rpm) for 90 min to remove the supernatant. A resulting pellet was resuspended in a virus storage solution and store at −80° C. for later use. Quantitative qPCR was conducted to detect lentivirus titers. The lentivirus had a titer of 4.8×10⁷ TU/mL.
2. Expression of PML-1 at the mRNA level in M1 inflammatory macrophages detected by RT-qPCR

Approximately 1×10⁶ THP-1 cells were inoculated in a 6-well plate, and PMA (propylene glycol methyl ether acetate) was added to reach a concentration of 30 ng mL⁻¹. After 72 h of culture, the medium was replaced and floating cells were completely removed, and a fresh medium (without PMA) was added to allow culture for 24 h to obtain MO macrophages. LPS was added to the RPMI-1640 medium containing 10% fetal calf serum to make an LPS concentration in the medium at 20 ng mL⁻¹, and then the MO macrophages were cultured for 48 h to induce polarization of the macrophages into an inflammatory phenotype (M1 macrophages). Several wells of M1 cells in the logarithmic growth phase were inoculated in a 24-well culture plate and cultured overnight in a 37° C., 5% CO₂ incubator. The old medium in the culture vessel was removed, the vessel was washed twice with PBS, and added with ½ volume of fresh medium. 100 μL of the lentivirus obtained in step 1 was added for infection, the virus liquid and culture medium were mixed, and filled up to a full culture volume after 4 h to continue the culturing. The virus was placed in a carbon dioxide incubator (37° C., 5% CO₂) and incubated for 48 h. The M1 macrophages were transfected with the lentivirus obtained experimentally in step 1. After the stably transfected cells reached the logarithmic growth phase, the cells were digested and collected.

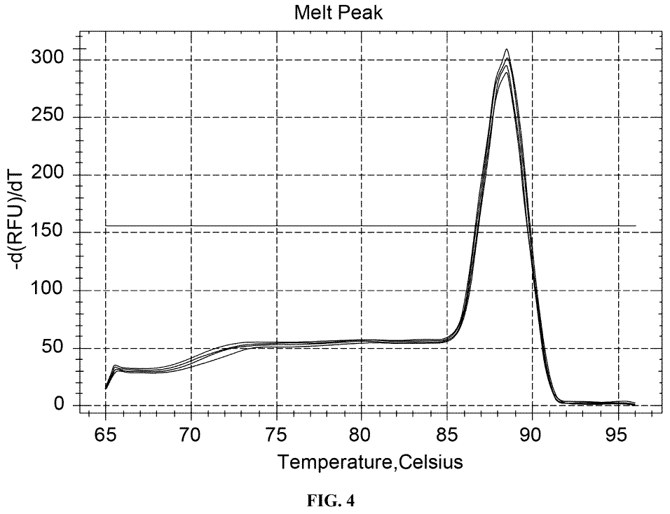

RNA was extracted from cells in the transfected group and control group using Trizol method, and reverse-transcribed into cDNA using a TRKARA reverse transcription kit. PCR amplification was conducted using the cDNA as a template with an SYBR Green Mix qPCR kit, and a dissolution curve was drawn (FIG. 4). The relative expression level of PML-1 mRNA in the M1 inflammatory macrophages was calculated by a 2−^{ΔΔct} method (Table 3), to detect overexpression efficiency of PML-1 in the M1 macrophages. The primer information used in qPCR was shown in Table 2, and a qPCR reaction system (20 μL) included: 10 μL of SYBR Premix Ex Taq, 0.4 μL each of upstream and downstream primers, 2 μL of cDNA template, and 7.2 μL of ddH₂O. A PCR program included: initial denaturation at 95° C. for 3 min; 23 cycles of denaturation at 95° C. for 15 s, annealing at 60° C. for 15 s, and extension at 72° C. for 15 s/kb; final extension at 72° C. for 5 min; and storage at 25° C.

TABLE 2
Primer information for qPCR

| Gene | Primer | | Primer | Fragment |
|---|---|---|---|---|
| name | name | Primer ID | sequence | size |
| hGAPDH | hGAPDH-F | SEQ ID | GAGTCCACTG | 123 bp |
| | | NO: 11 | GCGTCTTCAC | |
| | hGAPDH-R | SEQ ID | ATGGTTCACA | |
| | | NO: 12 | CCCATGACGA | |
| hPML-1 | hPML-1-F | SEQ ID | GACTTCTGGT | 219 bp |
| | | NO: 13 | GCTTTGAGTGC | |
| | hPML-1-R | SEQ ID | CTTGGAACATC | |
| | | NO: 14 | CTCGGCAGTA | |

TABLE 3
Detection results of qPCR on PML-1 expression level

| | (1) | (2) | ΔCT | ΔΔCT (Experimental | |
|---|---|---|---|---|---|
| Sample name | hPML-1 | hGAPDH | ((1) − (2)) | group-control group) | 2^{-ΔΔCT} |
| Overexpression | 22.20 | 18.87 | 3.33 | −5.58 | 47.96681 |
| group | | | | | |
| Control group | 27.12 | 18.21 | 8.91 | 0 | 1 |

As shown in Table 3, compared with the control group, the mRNA expression level of PML-1 in lentivirus-infected M1 inflammatory macrophages increased significantly; the expression level of hPML-1 gene in the overexpression group was 47.96681 times that of the control group. The above experimental results showed that lentivirus successfully infected the M1 inflammatory macrophages and expressed PML-1 protein; the transfection observation results showed that there was a desirable viral transduction effect of each experimental group, and the fluorescence rate reached not less than 80%.

### Example 3 Detection of PML-1 Protein Expression by Immunofluorescence and WB

### 1. Immunofluorescence Detection of PML-1 Expression in M1 Macrophages

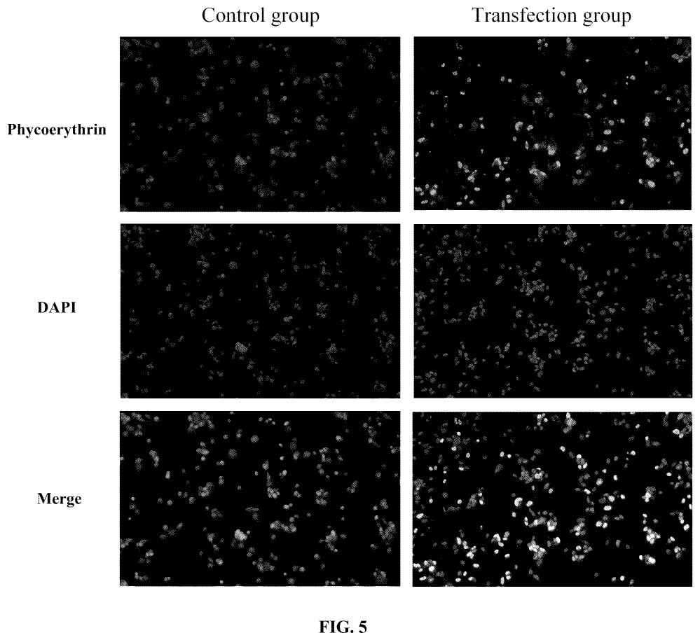

The medium was discarded from the transfected cells (i.e., the stably transfected M1 macrophages obtained in Example 2), the cells were washed 3 times with PBS, and fixated with 4% pre-cooled paraformaldehyde at room temperature for 15 min. After washing 3 times with pre-cooled PBS, 0.5% Tritonx-100 in PBS was added to allow permeabilization at room temperature for 20 min. After washing with pre-cooled PBS 3 times, normal goat serum was added to allow blocking at room temperature for 30 min, and rabbit anti-human PML monoclonal antibody (diluted at 1:200, purchased from Merck) was added to allow incubation overnight at 4° C. After washing 3 times with pre-cooled PBST, Phycoerythrin-goat anti-rabbit IgG (diluted at 1:200, purchased from Abcam) was added to allow incubation in the dark at 37° C. for 1 h. After washing 3 times with pre-cooled PBST, DAPI was added to allow incubation in the dark at 37° C. for 5 min. After washing, 1 drop of an anti-fluorescence quenching agent was added, the cells were sealed in a slide, and the expression of the target protein in the cells was observed under a fluorescence microscope. Meanwhile, the M1 cells transfected with empty vector were used as a control for immunofluorescence detection. The immunofluorescence detection results were shown in FIG. 5.

As shown in FIG. 5, the PML-1 lentiviral vector could produce specific orange-yellow fluorescence after infecting the M1 cells, and the fluorescence was mainly distributed in the nucleus. Although M1 cells transfected with empty vector could produce orange fluorescence, the fluorescence was weaker than that of the PML-1 lentivirus transfection group, and the difference was statistically significant (P<0.05). This indicated that M1 cells infected with PML-1 lentiviral vector had increased PML-1 protein expression.

### 2. Western Blotting (WB)

(1) Cell culture: approximately 1×10⁶ wild-type THIP-1 cells were inoculated into a 6-well plate, added with PMA (30 ng/mL), and cultured for 72 h. The medium was replaced, floating cells were completely removed, and a fresh medium (excluding PMA) was added to allow culture for 24 h. To generate M1 macrophages, LPS at a final concentration of 20 ng/mL was added to a 6-well plate and cultured in complete RPMI-1640 culture medium containing 10% fetal calf serum for 48 h to induce the polarization of macrophages toward an inflammatory M1 phenotype. The induced cells were divided into three experimental groups for three sets of experiments: (a) wild strain THP-1M1 induction; (b) wild strain THP-1M1 induction+lentivirus empty infection; (c) M1 type+lentiviral PML-1 infection. The specific lentivirus infection method was the same as step 2 in Example 2.
(2) Western blotting: the cells in the logarithmic growth phase were inoculated into 5 wells of a 6-well cell culture plate at 1×10⁶ cells/well, and cultured in a 37° C. incubator for 24 h. A RIPA lysis buffer was added to each well, the cells were lysed on ice for 20 min, then centrifuged at 12,000 rpm for 3 min to 5 min at 4° C., and transferred to a new EP tube. The protein concentration was measured using the BCA method, and then 5 times concentrated protein loading buffer (SDS buffer) was added according to the lysate volume, and boiled at 100° C. for 5 min. 20 g of protein samples were separated using 10% SDS polyacrylamide gel and 5% SDS concentration gel separately, the samples were transferred to PVDF membrane by electrophoresis, the PVDF membrane was blocked with 5% milk/TBST, the PVDF membrane was blocked with a shaker at room temperature for 1 h, and then incubated overnight with rabbit anti-human PML primary antibody. The membrane was incubated with HRP-labeled goat anti-rabbit secondary antibodies (purchased from Wuhan Sanying) for 1 h on a shaker at room temperature. Color development was conducted by a chemiluminescent gel imager (ChemiDoc™ XRS+, Bio-Rad, USA) using enhanced luminol reagent and ECL as chromogenic substrates, while rabbit anti-human 3-actin antibody (purchased from Wuhan Sanying) was used as an internal control.

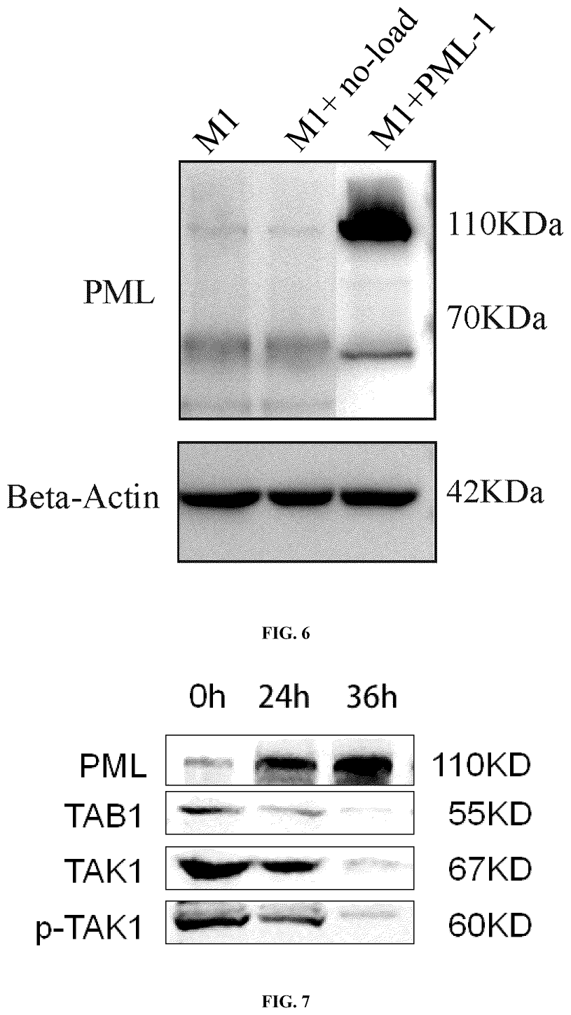

The Western blotting detection results were shown in FIG. 6.

As shown in FIG. 6, in the control (M1) and empty group, the expression level of PML-1 protein was low and showed a shallow and fuzzy band; while in the lentivirus infection group, the expression level of PML-1 protein increased significantly and showed dense bands. This indicated that the lentivirus successfully infected M1 macrophages and expressed the PML-1 protein.

Experimental Example 4 In vitro anti-cytokine storm effect of lentivirus expressing PML-1 protein

The cells were cultured according to the method in Example 3, and LPS was used to induce the polarization of the indicated cells toward an inflammatory phenotype (M1 macrophages). After LPS induction, the following experiment was conducted: 15×10⁶ cells were randomly divided into three groups, with 5×10⁶ cells in each group, and were infected with lentivirus for 0 h, 24 h, and 36 h, with 3 wells in each group. Western blotting was conducted according to the method in Example 3 to detect changes in TAB1, TAK1, and p-TAK1 proteins in the PML-1 and TLR4 signaling pathways.

The Western blotting detection results were shown in FIG. 7.

As shown in FIG. 7, as the lentivirus infection time increased in M1 macrophages, the PML-1 protein showed significantly increased content and formed dense bands; while the expression levels of TAB1, TAK1 and p-TAK1 decreased significantly, changing from dense and thick bands into light and fuzzy bands. This indicated that PML-1 had a desirable anti-inflammatory effect.

### Experimental Example 5 Anti-Inflammatory Effect of Lentivirus Expressing PML-1 (Inflammatory Cytokines)

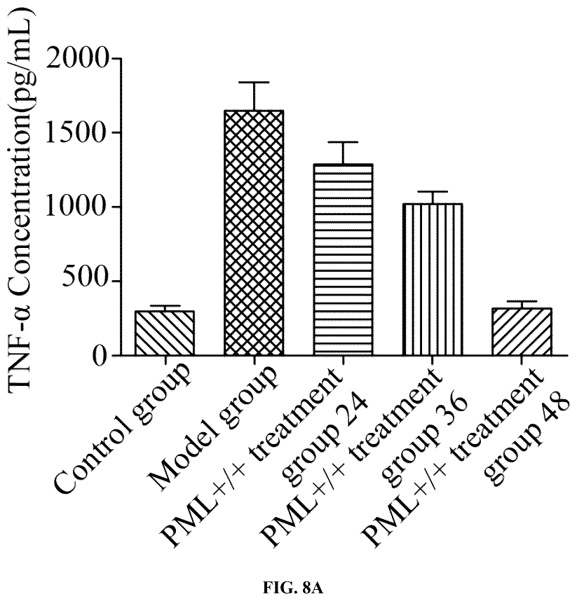

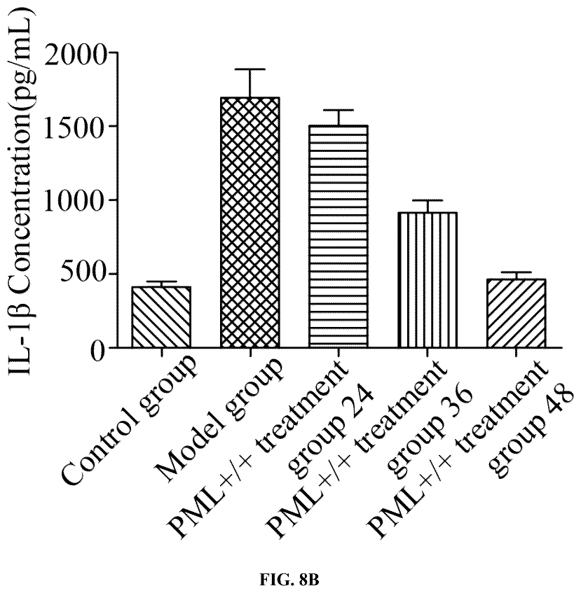

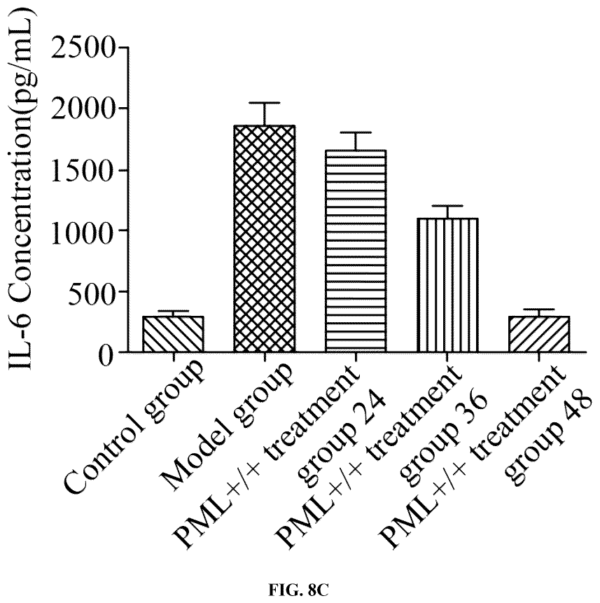

A THP-1 cell line was cultured according to the method in Example 3, and was induced by LPS for 48 h to polarize toward an inflammatory phenotype (M1 macrophages). After LPS induction, the following experiment was conducted: the cells were randomly divided into three groups, with 5×10⁶ cells in each group and 3 wells in each group, and the M1 macrophages were infected with lentivirus and treated for 24 h, 36 h, and 48 h, while a control group of THP-1 cells was established. The cells in each group were washed 2 times in RPMI medium, snap-frozen in liquid nitrogen, and thawed at 37° C. After repeated rapid freezing and thawing 2 times, cell debris was removed by centrifugation and a supernatant was remained. The corresponding cytokine expression levels in each supernatant sample were detected using human TNF-α, IL-1β, and IL-6 quantitative ELISA kits produced by ImmunoWay. The TNF-α, IL-1β, and IL-6 were detected in MO macrophages and M1 model group cells without lentivirus infection, which were used as the control group and model group, respectively. The detection results were shown in FIGS. 8A-C. In FIGS. 8A-C, the control group was detection results of MO macrophage-related inflammatory cytokines; the model group was detection results of cell-related inflammatory cytokines in the M1 model group without lentivirus infection. The PML treatment group 24 h, PML treatment group 36 h, and PML treatment group 48 h represented the inflammatory cytokine detection results of lentivirus-infected M1 macrophages treated for 24 h, 36 h, and 48 h, respectively.

As shown in FIGS. 8A-C, the anti-inflammatory effect of PML-1 was observed by detecting changes in cytokines using ELISA. In LPS-induced M1 inflammatory macrophages, the expression levels of inflammatory cytokines TNF-α, IL-1β, and IL-6 in M1 inflammatory macrophages increased dramatically compared with those in MO macrophage control group, showing characteristics of cytokine storm. After M1 inflammatory macrophages were treated with lentiviral infection, the expression levels of TNF-α, IL-1β, and IL-6 were significantly inhibited with the increase of lentiviral infection time, and decreased in a time-dependent manner. This indicated that the lentivirus expressing PML-1 protein could inhibit inflammatory cytokine storm.

### Example 6 In Vivo Anti-Cytokine Storm Effect of Lentivirus Expressing PML-1 Protein

1. Construction and Administration of Mouse Model with Acute Lung Injury Pneumonia

The mice were weighed and anesthetized with 1% sodium pentobarbital (50 mg/kg), fixed on the operating table in a supine position, their neck skin was gently wiped with iodophor and then disinfected with 75% alcohol cotton balls, a BALB/C mouse model was created, and LPS was injected into the trachea of mice into the lungs at a dose of 8 mg/kg, and the mice were shaken to make LPS enter the lungs as evenly as possible. 24 h after the first injection to create the model, LPS was injected again to create the model (LPS was injected into the trachea of mice into the lungs at a dose of 8 mg/kg, and the mice were shaken to make LPS enter the lungs as evenly as possible). After the modeling was completed, the mice were divided into 3 groups, with 8 mice in each group: (1) control group (referring to wild-type mice without any treatment and fed normally), (2) model group (referring to mice successfully modeled and fed normally), and (3) model treatment group.

In the model treatment group, 100 μL of lentivirus (titer 4.8×10⁷ TU/mL) was injected into the lungs of the mice to infect the mice. 5 d later, the mice in each group were anesthetized with sodium pentobarbital, blood samples were collected, and the animals were sacrificed by cervical dislocation. The left lung was fixated with paraformaldehyde and embedded in paraffin. The right lung was snap-frozen in liquid nitrogen and stored at −80° C. for ELISA.

### 2. Detection of TLR4 Signaling Pathway

The removed lung tissue was cut into pieces with ophthalmic scissors, added with RIPA containing protease inhibitor, ground, placed in a 5 mL EP tube, centrifuged at 12,000 r/min for 15 min at 4° C., and the samples were measured using a BCA protein quantification kit to obtain the protein concentration. The experimental method was shown in the Western blotting detection in Example 3, and the antibodies used in the experiment were shown in the table below.

The antibody information of the primary antibodies was shown in Table 4, and the antibody information of the secondary antibodies was shown in Table 5.

TABLE 4
Antibody information of primary antibodies

| | | Antibody | Dilution | Sampling |
|---|---|---|---|---|
| Name | Cat. No. | company | ratio | volume |
| PML | SAB5700065 | Merck | 1:1000 | 25 μg |
| p-TAK1 | YP0424 | Immunoway | 1:600 | |
| TAK1 | YT4536 | Immunoway | 1:1000 | |
| TAB1 | YM1302 | Immunoway | 1:1000 | |
| actin | 66009-1-Ig | Proteintech | 1:5000 | 5 μg |

TABLE 5
Antibody information of secondary antibodies

| Name | Dilution ratio |
|---|---|
| Goat anti-rabbit secondary antibody | 1:5000 |
| Goat anti-mouse secondary antibody | 1:5000 |

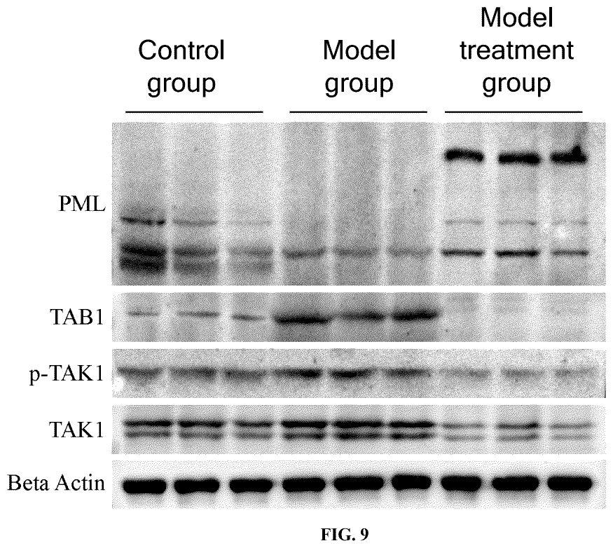

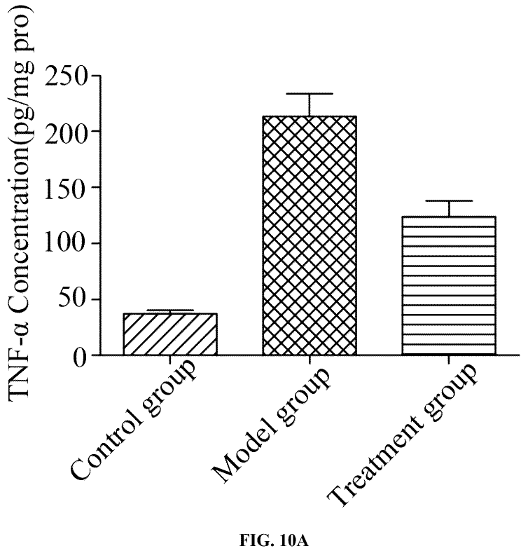

The Western blotting detection results were shown in FIG. 9.

It was observed through cell experiments that after PML-1 acted on M1 inflammatory macrophages, the PML-1 protein was activated and inhibited the TAB1, TAK1 and other proteins to achieve anti-inflammatory effects. In order to further verify the effect, LPS was used to prepare a mouse model with acute lung injury, as shown in FIG. 9. It was observed that after LPS stimulation, TAB1, TAK1, and p-TAK1 in the lung tissue of wild pneumonia mice were significantly increased compared with the control group, showing uniform and dense bands. However, no obvious changes in PML-1 protein were observed. After lentiviral treatment, PML-1 protein increased significantly and formed dense bands, while the expression levels of TAB1, TAK1, and p-TAK1 decreased significantly, changing from thick and dense bands to light and fuzzy bands. This indicated that lentiviral particles expressing PML-1 had excellent anti-inflammatory effects in vivo.

### 3. Detection of TNF-α, IL-1β, and IL-6

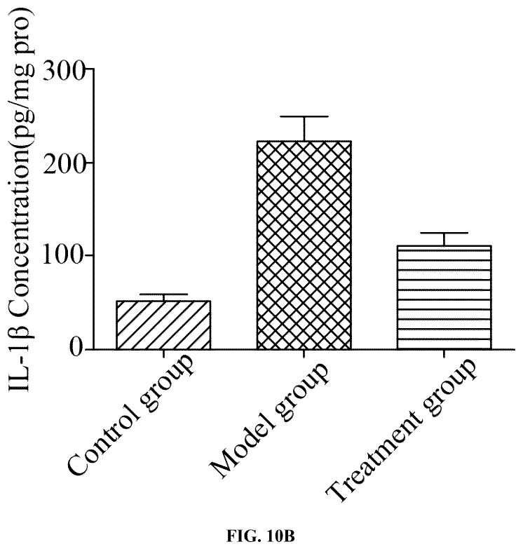

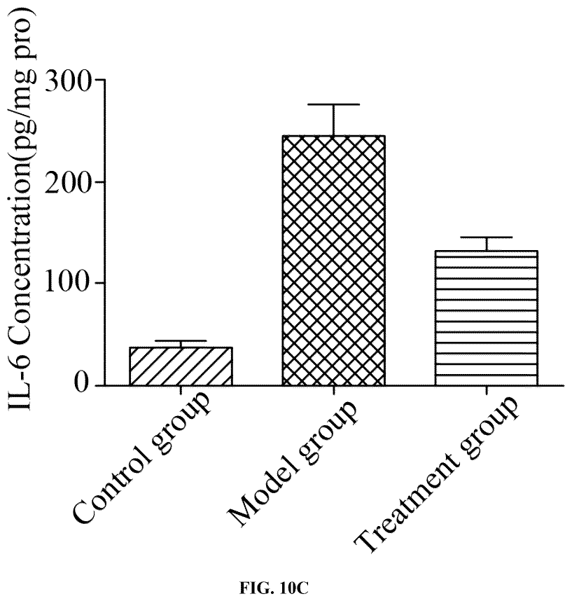

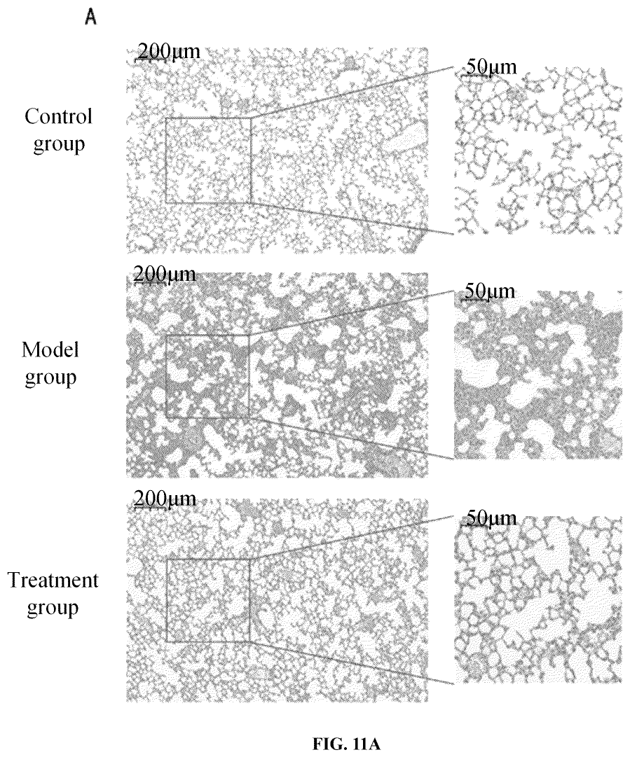

A quick-frozen lung tissue was thawed and weighed, and then 9 volumes of RIPA lysis buffer containing protein inhibitor cocktail III (Merck Millipore, USA) was added at a weight: volume ratio of 1 mg: 9 μL for grinding. The lysis buffer was centrifuged at 4° C. and 4,000 rpm for 10 min, and a supernatant was transferred to a clean centrifuge tube. 20 μL of each supernatant was diluted, and the protein concentration was determined using the BCA method. The remaining samples were placed in a refrigerator at 4° C. TNF-α, IL-1β, and IL-6 levels in plasma and lung tissue homogenates were measured using mouse ELISA kits (ImmunoWay Biotech, USA) according to the user manual. The detection results were shown in FIGS. 10A-C.

The anti-inflammatory effect of PML-1 in pneumonia mice was detected by ELISA method to detect changes in cytokines. The results are as shown in FIGS. 10A-C. In the LPS-induced acute lung injury mouse model, compared with the control group, the expression levels of inflammatory cytokines TNF-α, IL-1β, and IL-6 were significantly increased in pneumonia mice, showing characteristics of a cytokine storm. After treatment with lentivirus infection, the expression levels of TNF-α, IL-1β, and IL-6 in the lung tissue of pneumonia mice were significantly inhibited, indicating that lentivirus expressing PML-1 protein could still inhibit the inflammatory cytokine storm in vivo.

### 4. Pathological Section Observation

The left lung tissue was used for microscopic analysis of pathological changes. The lung tissue was fixated in 4% buffered formalin for 24 h, dehydrated, embedded in paraffin, sectioned, stained with hematoxylin and eosin, and analyzed on a Nikon Eclipse Ci microscope (Nikon Electronic Company, Osaka, Japan). 5 areas containing 300 alveoli were selected from each slide (10× zoom level of the image), and the extent of lung damage was assessed by an investigator who was blinded to the treatment group. The severity of lung inflammation was assessed semi-quantitatively according to the method described by Tanino Y et al (2002). Simply put, lung inflammation was rated on a scale of 0 to 5. Level 0, normal tissue; Level 1, mild inflammatory changes; Level 2, mild to moderate inflammatory changes (no obvious damage to lung structure); Level 3, moderate inflammatory damage (alveolar septal thickening); Level 4, moderate to severe inflammatory damage (forming nodules or areas of pneumonia that distorted normal structures); and Level 5, severe inflammatory injury, complete visual field occlusion. The images were collected and processed using Caseviewer 2.4 software.

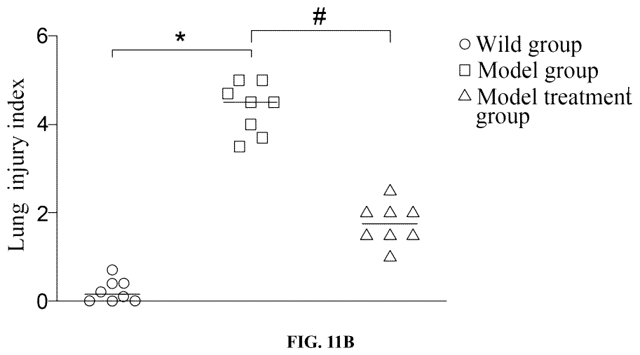

The results of histopathological analysis (H&E staining) of lung tissue of mice in each group were shown in FIGS. 11A-B, where a scale bar in the figure was 200 μm. The evaluation results of the degree of lung damage were shown in FIGS. 11A-B and Table 6.

TABLE 6
Evaluation results of lung injury degree

| | | | Model |
|---|---|---|---|
| Group | Wild group | Model group | treatment group |
| 1 | 0.7 | 3.5 | 2 |
| 2 | 0.4 | 4.7 | 2.5 |
| 3 | 0.1 | 3.7 | 1.5 |
| 4 | 0 | 5 | 1 |
| 5 | 0 | 4.5 | 1.5 |
| 6 | 0.2 | 4.5 | 2 |
| 7 | 0 | 5 | 1.5 |
| 8 | 0.4 | 4 | 2 |
| Average | 0.225 | 4.3625 | 1.75 |
| value | | | |
| SD | 0.254950976 | 0.570557372 | 0.46291005 |

As shown in FIGS. 11A-B and Table 6, the lung tissue of the control group showed a complete alveolar structure and alveolar spaces separated by a single layer of alveolar epithelial cells, and was transparent and clear. The lung tissue of mice in the LPS-induced wild model group showed severe pneumonia, which was characterized by: reduced alveolar space and significant thickening of the alveolar septa; there were large numbers of infiltrating neutrophils, macrophages, and other inflammatory cells, as well as large numbers of diffuse red blood cells; some exudates were also observed within the alveolar spaces. The microscopic injury severity index of mice in the LPS-induced model group was 4.36±0.57, which was significantly different from the wild mouse control group (0.23±0.25). Only mild inflammation, such as alveolar wall thickening and inflammatory cell infiltration, was observed in the model treatment group, and the inflammatory lesions were significantly lighter than those in the LPS model group, and there was no exudate in the alveolar cavity. The microscopic injury severity index was 1.75±0.46. The above experiments had proven that lentiviral particles expressing PML-1 protein could inhibit the expression of TAB1, TAK1, and p-TAK1 thereby inhibiting TLR-like inflammatory signaling pathways to achieve anti-inflammation.

### Implementation Example II

The PML-1 gene was inserted into the expression vector pcDNA3.4 (Detai Biotech) by whole-gene synthesis and double enzyme digestion. The accuracy of the final expression vector was confirmed by enzyme digestion and sequencing, and then the vector was transferred to the DH5a cloning strain. The transfection-grade plasmid was extracted using a plasmid extraction kit, and the plasmid was transfected into mammalian cells HEK293 using a transfection reagent for transient expression, and the hPML-1 protein was then purified by affinity chromatography.

The pcDNA3.4 used in the following technical scheme was modified pcDNA3.4, specifically: the GGATCTCTAGC base fragment (SEQ ID NO: 16) was inserted before GAATTC in the original pcDNA3.4 vector; the CCCGACCTCGAC base fragment (SEQ ID NO: 17) was inserted after GGATCC of the original pcDNA3.4 vector. For convenience, the modified pcDNA3.4 below was called pcDNA3.4.

### Example 1 Construction of PML-1 Overexpression Vector (Mammalian Recombinant Vector)

A vector expressing the human PML-1 gene under the control of the CMV promoter was created. Generally speaking: a target gene with restriction site was synthesized, a backbone vector was digested with a restriction endonuclease to obtain a linearized vector, which was ligated to the target gene. The overexpression vector was transformed, screened, and identified using conventional methods, and then used for subsequent transfection of mammalian cells after plasmid amplification and extraction. The flowchart of the experimental was shown in FIG. 1.
1. The PML-1 gene had a CDS sequence [NM 033238.3] shown in SEQ ID NO: 1, the sequence shown in SEQ ID NO: 1 was codon-optimized to obtain the nucleotide sequence shown in SEQ ID NO: 15, which is used for construction of mammalian recombinant vector. The whole-gene synthesis of the target gene was conducted using the nucleotide sequence shown in SEQ ID NO: 15.
2. An overexpression vector was constructed using the pcDNA3.4 vector as a backbone.
(1) The pcDNA3.4 vector was digested with BamHI/EcoRI restriction endonucleases (NEBs), where an enzyme digestion system included: 17.5 μL of Cutsmart, 600 ng to 800 ng of plasmid (pcDNA3.4 vector), 0.5 μL of BamHI, and 0.5 μL of EcoRI; the enzyme digestion was conducted at 37° C. for 1 h. After the enzyme digestion was completed, a gel was spotted and recovered, and a band was recovered using a gel recovery kit (NEB Monarch DNA) to obtain a linearized vector. The PML-1 gene and the linearized vector were ligated using a C115 Infusion ligase system (C115 enzyme from Vazyme Biotech), where the ligase systems included: 100 ng of linearized vector, 50 ng of target fragment, 5 μL of ligase, and making up to 10 μL with sterile water. The ligation was conducted at 50° C. and a hot lid temperature of 105° C. for 15 min. The recombinant plasmid was obtained after the reaction, namely the PML-1 overexpression vector.
3. After the ligation was completed, a plate (LB solid medium) with the corresponding resistance (Amp concentration of 100 g/mL) was dried in a 37° C. incubator upside down, while 500 μL of the antibiotic-free medium (LB culture medium) was preheated. The competent cells (DH5a competent cells) from −80° C. were placed on ice for 15 min to 20 min until melt, half of the ligation solution was added into the competent cells, flicked 5 times with fingers, and treated in ice bath for 30 min; the competent cells were quickly transferred to a preheated 42° C. water bath and heat-shocked for 1 min (heat shock for 45 s to 90 s with strict time control); when the heat shock was completed, the cells were immediately transferred to ice and placed for 2 min, then transferred to 500 μL of a preheated medium and cultured on a 37° C. shaker for 1 h; a bacterial solution was centrifuged at 4,000 rpm for 2 min to remove a supernatant (400 μL of the supernatant was discarded); about 20 glass beads were poured on the plate, the bacterial solution were mixed well by pipetting and then spread onto the plate with corresponding resistance (LB solid medium with Amp concentration of 100 g/mL), and cultured in a 37° C. incubator overnight.

4. Bacterial examination and extraction of recombinant plasmid (PML-1 overexpression vector)

The ultraviolet of an ultra-clean bench was turned on; after 20 min, the ultraviolet was turned off while the third wind speed was started, the ultra-clean bench was wiped with 75% ethanol to start the bacterial examination. On the ultra-clean bench, 25 μL of sterilized water was added to the bottom of a 96-well PCR plate, it was checked if there was air at the bottom against the light; the monoclonal plaques on the overnight culture plate were gently dipped with a sterilized 10 L white pipette tip, transferred into the corresponding 96-well reaction plate, mixed well by pipetting with a 10 μL row pipet 2 to 3 times, and 4 μL of samples were pipetted row by row to a new 96-well plate as a template to allow PCR reaction for bacterial examination. 50 μL of antibiotic-free broth medium was added to the original 96-well plate, covered by the sterilized 96-well plate and placed in a 30° C. incubator.

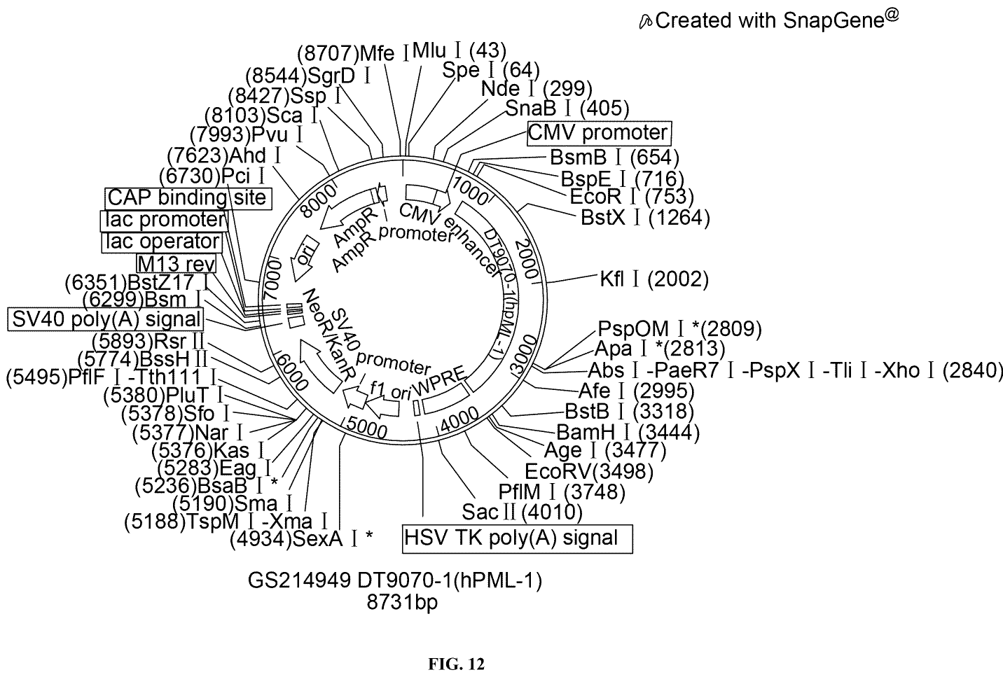

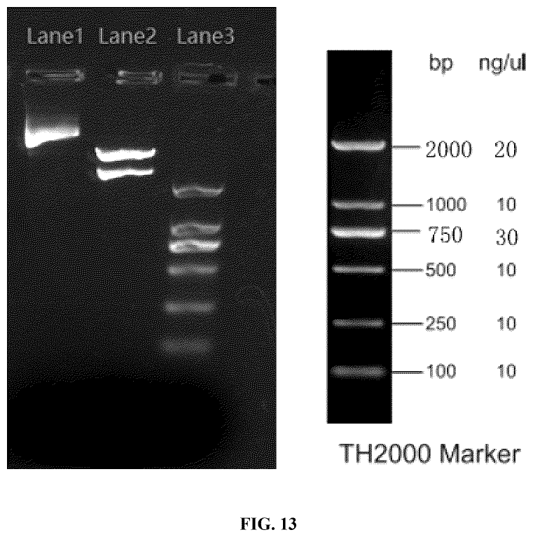

The clones that were positive by agarose gel electrophoresis were selected to allow sequencing verification to confirm that the recombinant vector containing the PML-1 target gene was obtained, indicating that the overexpression vector was constructed. A structure diagram of the overexpression vector was shown in FIG. 12.

A plasmid of the positive recombinant strain was extracted to obtain a recombinant plasmid, which was then identified by enzyme digestion.

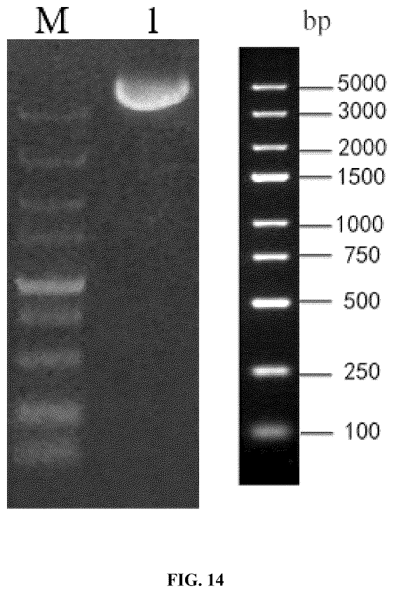

Based on the existing endonucleases, NTI/SnapGene was used to search for available restriction enzyme digestion plans to identify whether the plasmid was correct, where enzyme digestion system and conditions were the same as those in step (2). FIG. 13 showed the enzymatic digestion identification results of a recombinant plasmid. In FIG. 13, Lane1 corresponded to the complete plasmid; Lane2 corresponded to digestion reaction products of the EcoRI/BamHI restriction endonucleases; and Lane3 corresponded to marker.

The enzymatic digestion identification results showed that the obtained recombinant plasmid was the PML-1 overexpression vector.

### Example 2 Expression and Purification of hPML-1 Protein in Mammalian System

1. Transformation of DH5a with hPML-1 Plasmid

80 ng to 100 ng of the expression plasmid containing the hPML-1 gene (PML-1 overexpression vector or mammalian recombinant vector) was aspirated to the prepared DH5a competent cells. The transformed competent cells were cultured in an LB liquid medium on a shaker at 37° C. and 200 rpm for about 30 min. A portion of the suspension was spread on a plate containing ampicillin resistance and cultured in an incubator at 37° C. overnight. A single clone was selected and cultured in 2 mL to 5 mL of LB medium at 37° C. and 200 rpm for 8 h. The cells were inoculated into 200 mL of LB medium at a volume ratio of 1/500 and cultured at 37° C. and 200 rpm for 16 h. The cultured bacterial solution was collected and centrifuged to remove the supernatant. A transfection-grade plasmid, namely PML-1 overexpression plasmid, was extracted using a plasmid extraction kit. Agarose gel analysis was conducted on the plasmid, and the results were shown in FIG. 14. In FIG. 14, M represented a DNA marker, and only one electrophoresis band was shown in Lane 1, indicating that the PML-1 overexpression plasmid was successfully extracted.

2. Transfection of hPML-1 Plasmid into HEK293 Cells

1 d before transfection, the HEK293 cells were cultured in suspension to 500 mL, with an inoculation density of 1×10⁶ cells/mL, and incubated in an incubator at 110 rpm, 37° C., and 5% CO₂. The cell density was controlled at about (1−1.5)×10⁶ cells/mL. A DNA-transfection reagent mixture (a product of transfection-grade plasmids extracted by the plasmid extraction kit and the transfection reagent mixed together in a certain proportion) into the cells to be transfected, and incubated in the incubator at 110 rpm, 37° C., 5% CO₂. About 4 d to 6 d after transfection, the cell culture was taken out and centrifuged to collect a supernatant or cells.

3. Purification of hPML-1 Protein

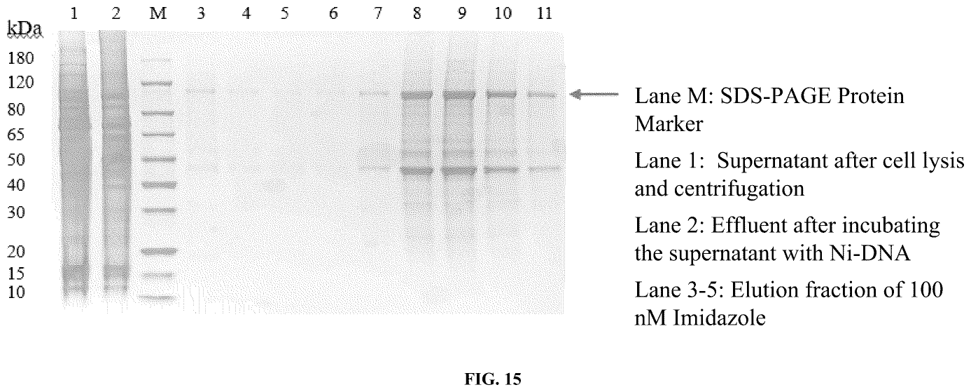

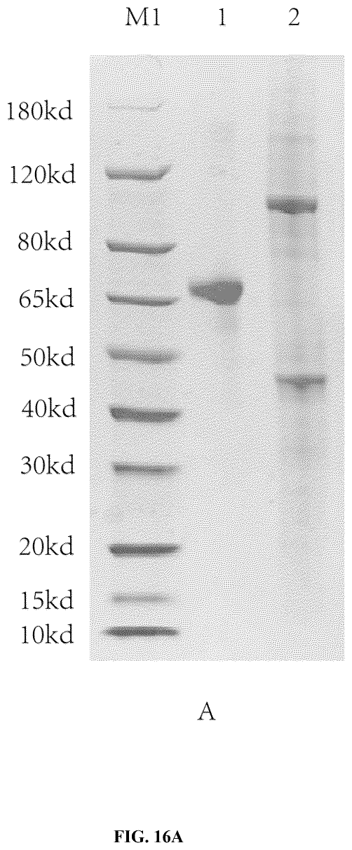

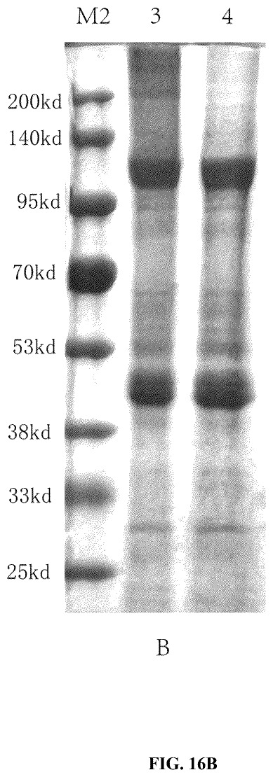

After 5 d of transfection and culture, the cell medium was centrifuged and cells obtained were dissolved in 50 mM Tris, 300 mM NaCl, 8 M Urea, 20 mM Imidazole, and pH=8.0 buffer, purified using a Ni-IDA column, and then subjected to SDS-PAGE analysis. After purification by Ni-IDA affinity chromatography, a purified group with relatively high purity was collected and added to a treated dialysis bag, and dialyzed into a buffer [1×PBS (pH=7.4), 4 mM GSH, 0.4 mM GSSG, 0.4 M L-Arginine, 1 M Urea] at 4° C. for renaturation, and a renatured hPML-1 protein was finally dialyzed in a stock solution of 1×PBS, pH=7.4 for about 6 h to 8 h. After dialysis and renaturation, a supernatant was filtered with a 0.22 m filter, aliquoted, and frozen at −80° C. for later use. The SDS-PAGE analysis results of the PML-1 recombinant protein were shown in FIG. 15 and FIG. 16A.

As shown in FIG. 15 and FIG. 16A, the PML-1 target band with a molecular weight of 120 KD was successfully isolated in Lanes 8 to 11 through Ni-IDA column purification.

4. Quality inspection of hPML-1 protein by Western blotting

The HEK293 cells in the logarithmic growth phase were inoculated into 5 wells of a 6-well cell culture plate at 1×10⁶ cells/well, and cultured in a 37° C. incubator for 24 h (the medium was RPMI 1640 supplemented with 10% fetal bovine serum). A RIPA lysis buffer was added to each well, the cells were lysed on ice for 20 min, then centrifuged at 12,000 rpm for 3 min to 5 min at 4° C., and transferred to a new EP tube. The protein concentration was measured using the Bradford method, and then 5 times SDS buffer was added according to the lysate volume, and boiled at 100° C. for 5 min. 20 g of protein samples were separated using 10% SDS polyacrylamide gel and 5% SDS concentration gel separately, the samples were transferred to PVDF membrane by electrophoresis, the PVDF membrane was blocked with 5% milk/TBST, the PVDF membrane was blocked with a shaker at room temperature for 1 h, and then incubated overnight with His-tagged mouse monoclonal primary antibody. The membrane was incubated with HRP-labeled goat anti-mouse secondary antibodies (purchased from Wuhan Sanying) for 1 h on a shaker at room temperature. Color development was conducted by a chemiluminescent gel imager using enhanced luminol reagent and ECL as chromogenic substrates, while rabbit anti-human 3-actin antibody was used as an internal control. The detection results were shown in FIG. 16B. In FIGS. 16A-B, 1 represented the BSA control, and 2, 3, and 4 represented PML-1 protein.

As shown in FIGS. 16A-B, the PML-1 protein had two bands, one main band and one secondary band. When the PML-1 gene was expressed, two transcripts were produced; a complete PML-1 gene transcript could express a functional PML-1 protein body, with a molecular weight of approximately 110 KD; another transcript, a copy produced during the editing of PML-1, was located at a C-terminus outside an RBCC domain of the PML-1 gene, with an expressed molecular weight of approximately 45 KD. This indicated that the position of the Western blotting band of PML-1 protein was exactly the same as the SDS-PAGE detection result.

### Example 3 Anti-Inflammatory Effect of PML-1 Recombinant Protein

### 1. Detection of TLR4 Signaling Pathway

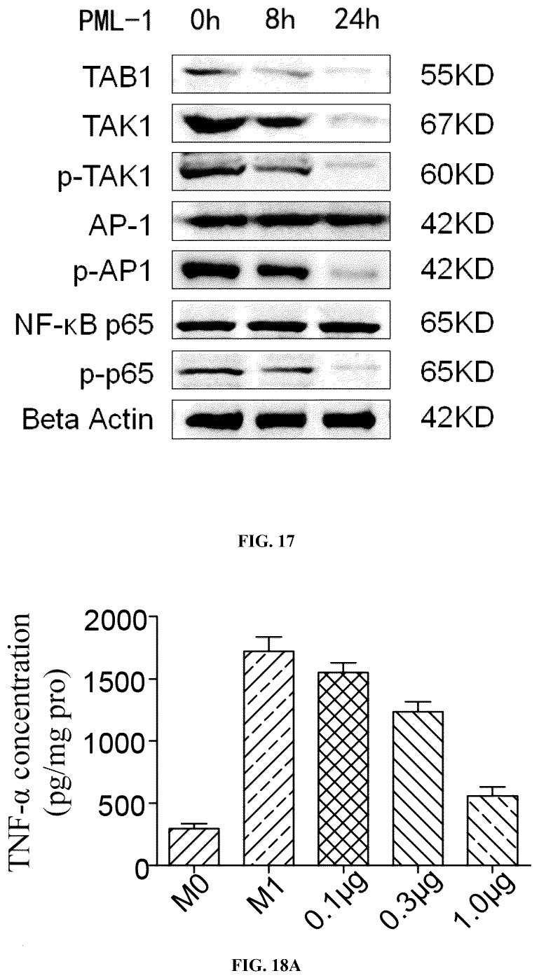

Approximately 1×10⁶ wild-type THP-1 cells were inoculated into a 6-well plate, added with PMA (30 ng/mL), and cultured for 72 h. The medium was replaced, floating cells were completely removed, and a fresh medium (excluding the PMA) was added to allow culture for 24 h. To generate M1 macrophages, LPS at a final concentration of 20 ng/mL was added to a 6-well plate and cultured in complete RPMI-1640 culture medium containing 10% fetal calf serum for 48 h to induce the polarization of macrophages toward an inflammatory (M1) phenotype. After the LPS induction was completed, the following experiments were conducted: three groups of experiments were conducted, 5×10⁶ cells were taken from each group of experiments and treated with PMIL-1 recombinant protein at a concentration of 0.4 g/mL for 0 h, 8 h, and 24 h, with 3 wells in each group. Western blotting was conducted according to the method in Example 2 to detect changes in TAB1, TAK1, p-TAK1, p-AP1, p-P65, APi, and P65 proteins in the TLR4 signaling pathway. The detection results were shown in FIG. 17.

As shown in FIG. 17, as the action time of PML-1 increased, the expression levels of TAB1, TAK1, and p-TAK1 decreased significantly in M1 macrophages, changing from thick and dense bands to light and fuzzy bands. At the same time, it was observed that PML-1 protein had no regulatory effect on AP1 and P65, but only regulated their respective phosphorylation. In M1 macrophages, both API and P65 were uniform dark bands; after 8 h and 24 h of PML-1 protein treatment, the protein bands of AP1 and P65 showed no significant changes compared with 0 h. In M1 macrophages, both p-APi and p-P65 decreased as the time of exposure to PML-1 protein increased; after 24 h of treatment, the protein bands appeared as light and fuzzy bands. This indicated that the PMIL-1 recombinant protein could inhibit the TAB1/TAK1 pathway protein of the TLR inflammatory signaling pathway.

### Experimental Example 4 In Vitro Anti-Cytokine Storm Effect of hPML-1

### Detection of Inflammatory Cytokines

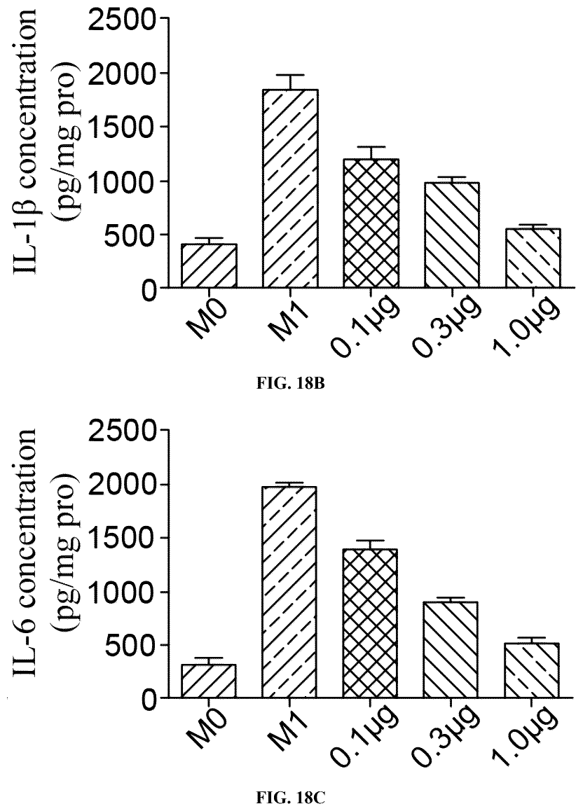

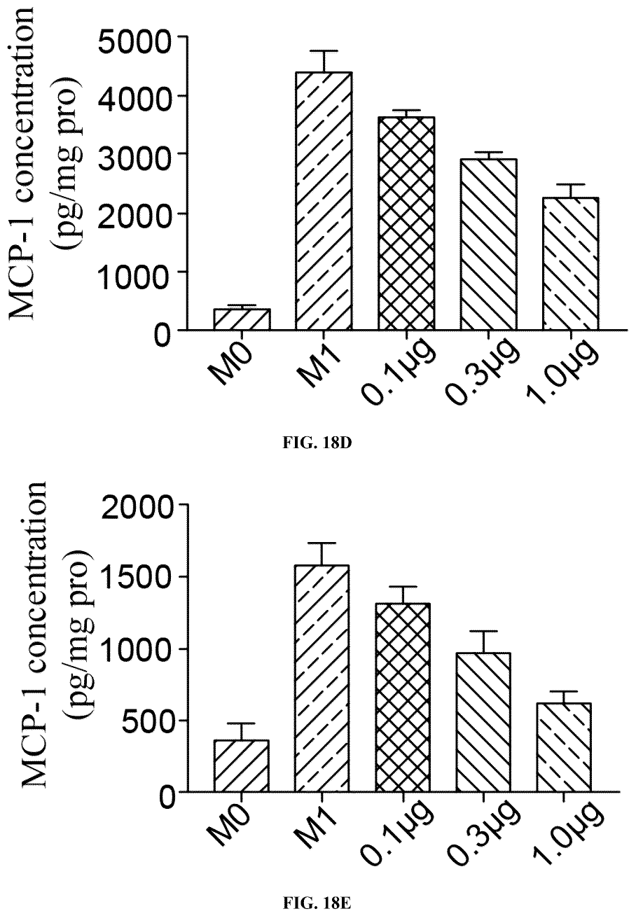

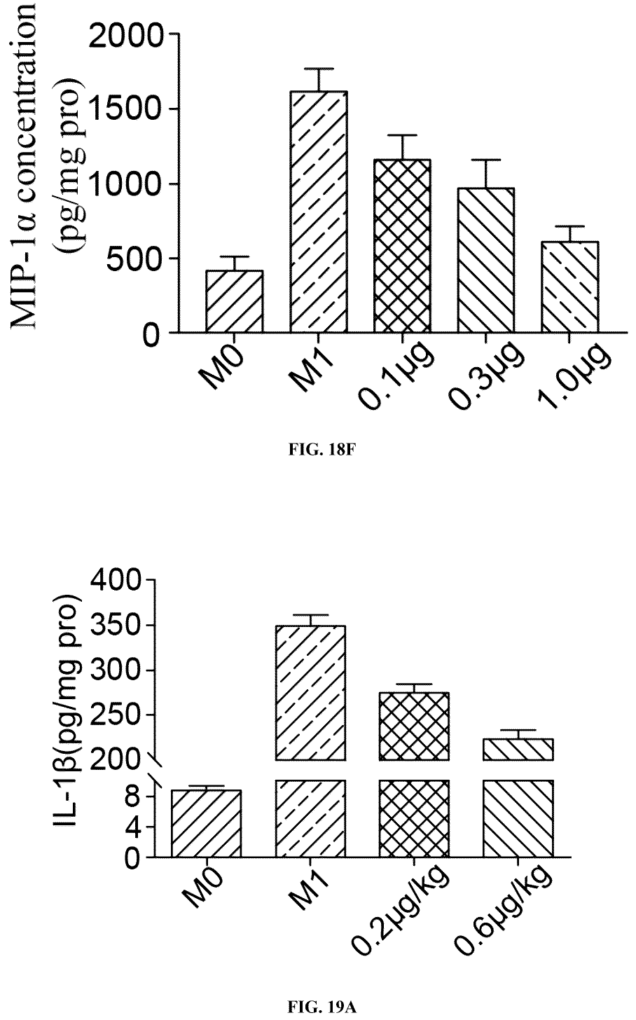

The cells were cultured according to the method in Example 3, and polarization of macrophages toward an inflammatory phenotype (M1 macrophages) was induced. After induction, the cells were treated with PMIL-1 protein at 0 g mL⁻¹, 0.1 g mL⁻¹, 0.3 g mL⁻¹, and 1.0 g mL-1 for 24 h. 5×10⁶ cells were taken from each group, with 3 wells in each group, and an MO macrophage control group was established. The cells in each group were washed 2 times in RPMI medium, snap-frozen in liquid nitrogen, and thawed at 37° C. After repeated rapid freezing and thawing 2 times, cell debris was removed by centrifugation and a supernatant was remained. The corresponding cytokine expression levels in each supernatant sample were detected using human TNF-α, IL-1β, MIP-1α, IL-6, IL-8, and MCP-1 quantitative ELISA kits produced by ImmunoWay. The detection results of inflammatory cytokines were shown in FIGS. 18A-F.

As shown in FIGS. 18A-F, the PML-1 recombinant protein had a significant inhibitory effect on the expression of inflammatory cytokines (TNF-α, IL-1, IL-6, IL-8, MIP-1α, and MCP-1) in M1 macrophages induced by LPS. (1) The expression of inflammatory cytokines TNF-α, IL-1β, IL-6, IL-8, MIP-1α, and MCP-1 was low in MO macrophages; however, after LPS induction, these cytokines showed an explosive increase and showed the characteristics of cytokine storm. For example, TNF-α had an expression level of 279.96±61.08 pg·mL⁻¹ in MO macrophages; after polarization into M1 macrophages, the expression level increased sharply to 1721.50±241.81pg·mL⁻¹. (2) When PML-1 protein was administered at doses of 0.1 g mL⁻¹, 0.3 g mL⁻¹, and 1.0 g mL⁻¹ for 24 h, the expression levels of these inflammatory cytokines were decreased in a dose-dependent manner, where the levels of TNF-α, IL-6, and IL-1β decreased most significantly. For example, as the dose of PML-1 increased, the expression levels of TNF-α were 1553.00±160.76 pg·mL⁻¹, 1237.05±172.32 pg·mL⁻¹, and 563.29±151.29 pg·mL⁻¹, respectively, showing a trend of significant and gradual decrease. Compared with the expression level of the untreated model group, except for the 0.1 g mL⁻¹ dose group, the expression levels of the other groups were statistically different (P<0.05).

IL-1β had an expression level of 406.25±113.69 pg·mL⁻¹ in MO macrophages; after polarization into M1 macrophages, the expression level increased sharply to 1839.25±283.24 pg mL⁻¹. When PML-1 protein was administered at doses of 0.1 g mL⁻¹, 0.3 g mL⁻¹, and 1.0 g mL⁻¹ for 24 h, the expression levels of these inflammatory cytokines decreased in a dose-dependent manner. The expression levels were 1196.66+231.93 pg·mL⁻¹, 976.29±95.81 pg·mL⁻¹, and 548.38±71.31 pg·mL⁻¹, respectively, showing an obvious gradual downward trend. IL-6 had an expression level of 314.69±117.49 pg·mL⁻¹ in MO macrophages; after polarization into M1 macrophages, the expression level increased sharply to 1970.32±52.69 pg·mL⁻¹. When PML-1 protein was administered at doses of 0.1 g mL⁻¹, 0.3 g mL⁻¹, and 1.0 g mL⁻¹ for 24 h, the expression levels of these inflammatory cytokines decreased in a dose-dependent manner. The expression levels were 1386.59±169.67 pg·mL⁻¹, 895.34+68.63 pg·mL⁻¹, and 508.83±100.51 pg mL⁻¹, respectively, showing an obvious gradual downward trend.

IL-8 had an expression level of 351.40±63.38 pg·mL⁻¹ in MO macrophages; after polarization into M1 macrophages, the expression level increased sharply to 4387.66±370.29 pg mL⁻¹. When PML-1 protein was administered at doses of 0.1 g mL⁻¹, 0.3 g mL⁻¹, and 1.0 g mL⁻¹ for 24 h, the expression levels of these inflammatory cytokines decreased in a dose-dependent manner. The expression levels were 3627.95±120.61 pg·mL⁻¹, 2911.23±116.27 pg·mL⁻¹, and 2258.14±219.72 pg·mL⁻¹, respectively, showing an obvious gradual downward trend.

MCP-1 had an expression level of 361.00±116.89 pg·mL⁻¹ in MO macrophages; after polarization into M1 macrophages, the expression level increased sharply to 1576.71±154.95 pg mL⁻¹. When PML-1 protein was administered at doses of 0.1 g mL⁻¹, 0.3 g mL⁻¹, and 1.0 g mL⁻¹ for 24 h, the expression levels of these inflammatory cytokines decreased in a dose-dependent manner.

The expression levels were 1311.83±117.79 pg·mL⁻¹, 969.83±151.52 pg·mL⁻¹, and 617.97+85.98 pg mL⁻¹, respectively, showing an obvious gradual downward trend.

MIP-la had an expression level of 415.84±91.01 pg·mL⁻¹ in MO macrophages; after polarization into M1 macrophages, the expression level increased sharply to 1622.64±144.10 pg mL⁻¹. When PML-1 protein was administered at doses of 0.1 g mL⁻¹, 0.3 g mL⁻¹, and 1.0 g mL⁻¹ for 24 h, the expression levels of these inflammatory cytokines decreased in a dose-dependent manner. The expression levels were 1161.74±163.45 pg·mL⁻¹, 970.71±188.97 pg·mL⁻¹, and 608.68±101.84 pg·mL⁻¹, respectively, showing an obvious gradual downward trend.

Example recombination 5 In vivo anti-cytokine storm effect of PML-1 recombinant protein

Construction and Administration of Mouse Model with Acute Lung Injury Pneumonia

1. The modeling was as shown in Example 6 of Implementation Example I. After the modeling was completed, the following experiments were conducted: 0 μg·kg⁻¹, 0.2 μg·kg⁻¹, and 0.6 g kg⁻¹ of PML-1 protein were intraperitoneally injected for 24 h; after treatment, mice in each group were anesthetized with sodium pentobarbital, blood samples were collected, and then the mice were killed by cervical dislocation. The left lung was fixated with paraformaldehyde and embedded in paraffin. The right lung was snap-frozen in liquid nitrogen and stored at −80° C. for ELISA.

### 2. Detection of Inflammatory Cytokines

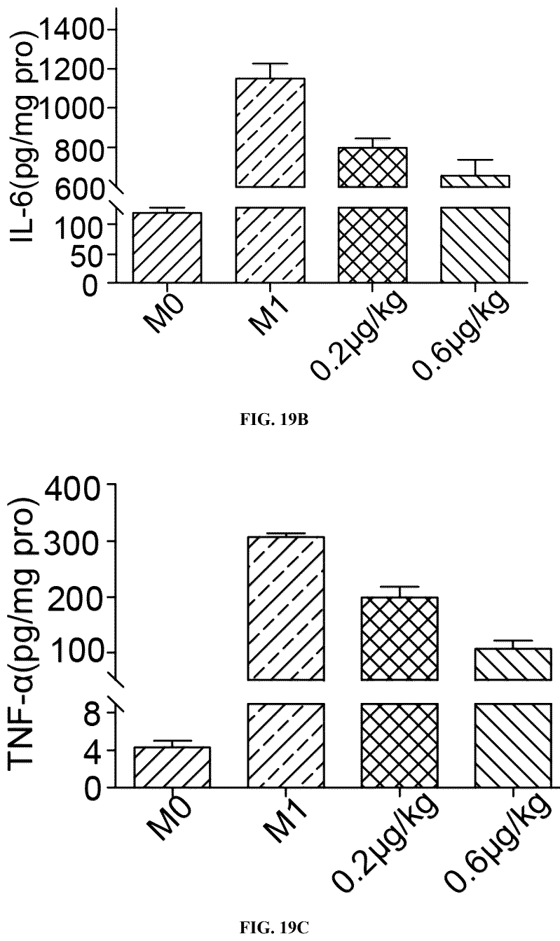

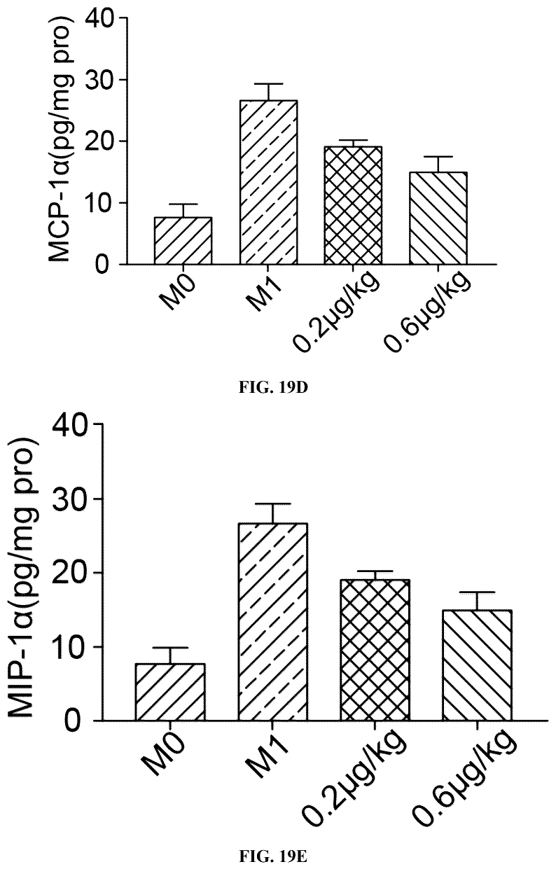

A quick-frozen lung tissue was thawed and weighed, and then 9 volumes of RIPA lysis buffer containing protein inhibitor cocktail III (Merck Millipore, USA) was added at a weight (mg): volume (L) ratio of 1:9 μL for grinding. The lysis buffer was centrifuged at 4° C. and 4,000 rpm for 10 min, and a supernatant was then transferred to a clean centrifuge tube. 20 μL of each supernatant was diluted, and the protein concentration was determined using the BCA method. The remaining samples were placed in a refrigerator at 4° C. TNF-α, IL-1β, MIP-1α, IL-6, and MCP-1 levels in plasma and lung tissue homogenates were measured using mouse ELISA kits (ImmunoWay Biotech, USA) according to the user manual. The results of the anti-cytokine storm effect in vivo were shown in FIGS. 19A-E. MO referred to wild-type mice, M1 referred to mice in the model group or mice treated with 0 μg·kg⁻¹ PML-1 protein via intraperitoneal injection; 0.2 μg·kg⁻¹ and 0.6 μg·kg⁻¹ represented mice treated with 0.2 μg·kg⁻¹ PML-1 protein via intraperitoneal injection and mice treated with 0.6 μg·kg⁻¹ PML-1 protein via intraperitoneal injection, respectively.

The anti-inflammatory effect of PML-1 in pneumonia mice was detected by ELISA method to detect changes in cytokines. As shown in the LPS-induced acute lung injury mouse model of FIGS. 19A-E, compared with the control group, the expression levels of inflammatory cytokines TNF-α, IL-1β, IL-6, MCP-1, and MIP-la were significantly increased in pneumonia mice, showing characteristics of a cytokine storm. As the dose of PML-1 increased, the expression level of the above cytokines in the lung tissue of pneumonia mice decreased in a dose-dependent manner, indicating that PML-1 protein could still inhibit inflammatory cytokine storm in vivo.

### 3. Pathological Section Observation

The steps were shown in Example 6 of Implementation Example I. The pathological sections and the evaluation results of the degree of lung damage were shown in FIGS. 18A-F, and the lung inflammation score results were shown in Table 7. The model treatment group referred to the treatment group in which 0.6 μg·kg¹ PML-1 protein was injected intraperitoneally.

TABLE 7
Score results of pulmonary inflammation

| | | | Model |
|---|---|---|---|
| Group | Wild group | Model group | treatment group |
| 1 | 0.7 | 3.1 | 3 |
| 2 | 0.4 | 4.5 | 1 |
| 3 | 0.1 | 3.5 | 1.5 |
| 4 | 0.1 | 4.5 | 1.5 |
| 5 | 0 | 5 | 1.5 |
| 6 | 0.1 | 4.5 | 2.5 |
| 7 | 0 | 5 | 1.5 |
| 8 | 0.3 | 4 | 1.5 |
| Average | 0.2125 | 4.2625 | 1.75 |
| value | | | |
| SD | 0.23687784 | 1.557597866 | 0.845740964 |

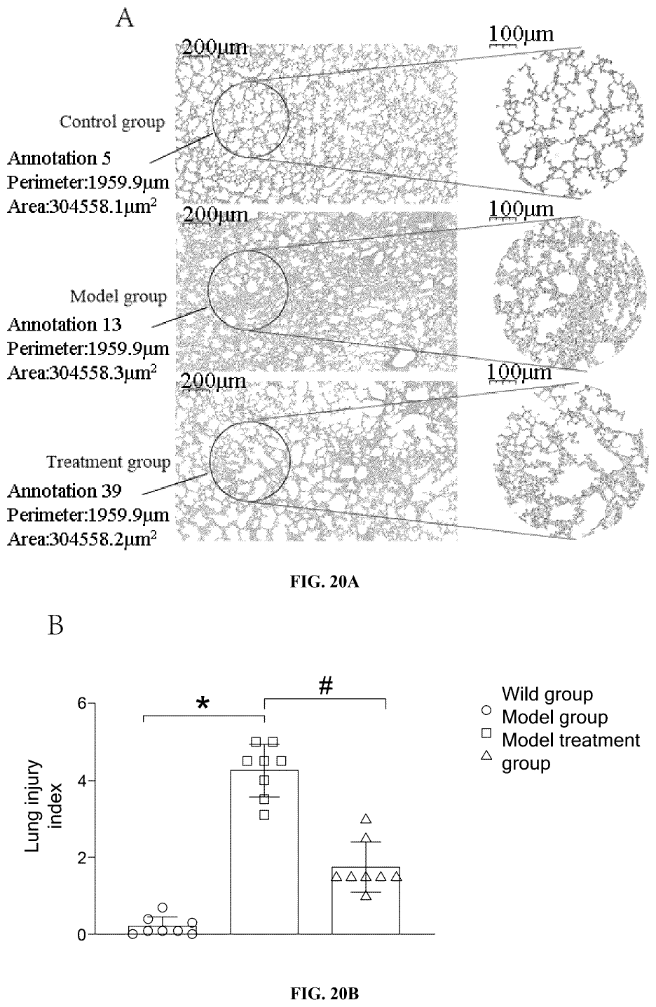

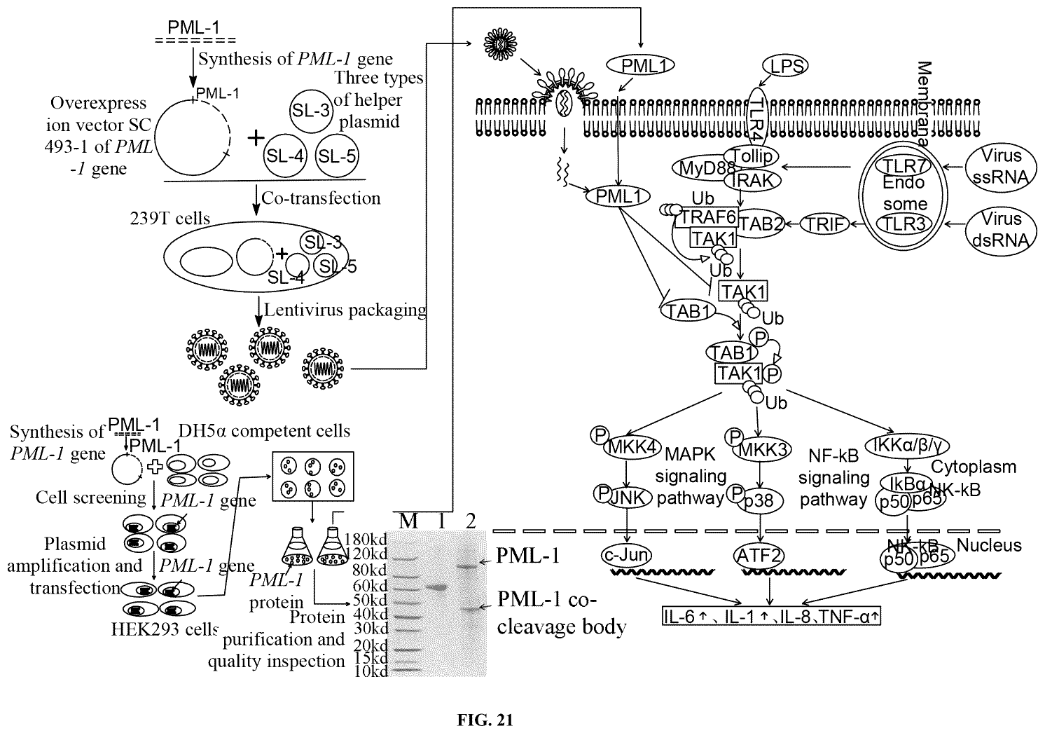

The results of histopathological analysis (H&E staining) of lung tissue of mice in each group were shown in FIGS. 20A-B. The lung tissue of the control group showed a complete alveolar structure and alveolar spaces separated by a single layer of alveolar epithelial cells, and was transparent and clear. The lung tissue of mice in the LPS-induced wild model group showed severe pneumonia, which was characterized by: reduced alveolar space and significant thickening of the alveolar septa; there were large numbers of infiltrating neutrophils, macrophages, and other inflammatory cells, as well as large numbers of diffuse red blood cells; some exudates were also observed within the alveolar spaces. The microscopic injury severity index of mice in the LPS-induced model group was 4.26±1.56, which was significantly different from the wild mouse control group (0.21+0.24). Only mild inflammation, such as alveolar wall thickening and inflammatory cell infiltration, was observed in the wild mouse model treatment group, and the inflammatory lesions were significantly lighter than those in the LPS model group, and there was no exudate in the alveolar cavity. The microscopic injury severity index was 1.75±0.85.

In summary, the PML-1 expression preparation provided by the present disclosure can significantly inhibit the expression of proteins TAB1, TAK1, and p-TAK1 and significantly inhibit inflammatory cytokines TNF-α, IL-1β, MIP-1α, IL-6, IL-8, and MCP-1 in an inflammatory signaling pathway, thereby inhibiting the inflammatory cytokine storm. The PML-1 expression preparation is particularly suitable for inhibiting cytokine storm and inflammatory storm caused by viral infections such as COVID-19, severe influenza, and AIDS. Other diseases with the cytokine storm as an endpoint, such as systemic lupus erythematosus and rheumatoid arthritis, are also desirable indications of the protein. The protein can also be used in neurodegenerative diseases caused by bacterial and viral infections, including Alzheimer's disease, Parkinson's disease, multiple sclerosis, and Huntington's disease.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A method for using a promyelocytic leukemia 1 (PML-1) protein and/or a product expressing the PML-1 protein, comprising the following processes:
(1) a process of inhibiting a cytokine storm using the PML-1 protein and/or the product expressing the PML-1 protein;
(2) a process of conducting anti-inflammation using the PML-1 protein and/or the product expressing the PML-1 protein;
(3) a process of treating a disease with the cytokine storm as a main pathological feature using the PML-1 protein and/or the product expressing the PML-1 protein;
(4) a process of regulating an inflammatory signaling pathway using the PML-1 protein and/or the product expressing the PML-1 protein; and
(5) a process of regulating an inflammatory cytokine using the PML-1 protein and/or the product expressing the PML-1 protein; wherein
the PML-1 protein has an amino acid sequence shown in SEQ ID NO: 3.

2. The method according to claim 1, wherein the disease is one or more selected from the group consisting of pneumonia, an autoimmune disease, a tumor, AIDS, and a neurodegenerative disease; and
the inflammatory cytokine is one or more selected from the group consisting of TNF-α, IL-1β, IL-6, IL-8, MIP-1α, and MCP-1.

3. The method according to claim 1, wherein the PML-1 protein is encoded with a nucleotide sequence shown in SEQ ID NO: 1 or SEQ ID NO: 15.

4. The method according to claim 1, wherein the product comprises a recombinant vector.

5. The method according to claim 4, wherein the recombinant vector is selected from the group consisting of a lentiviral recombinant vector and a mammalian recombinant vector.

6. A recombinant vector expressing a PML-1 protein, comprising a base vector and a nucleotide sequence encoding the PML-1 protein; wherein
the base vector is selected from the group consisting of a lentiviral base vector and a mammalian base vector.

7. The recombinant vector according to claim 6, wherein the lentiviral base vector uses an elongation factor 1 alpha (EF1A) as a promoter; and
the mammalian base vector comprises pcDNA3.4.

8. A preparation method of a PML-1 protein and/or a product expressing the PML-1 protein, comprising expressing the recombinant vector according to claim 6.

9. A preparation method of a PML-1 protein and/or a product expressing the PML-1 protein, comprising expressing the recombinant vector according to claim 7.

10. The preparation method according to claim 8, wherein when the recombinant vector is a lentiviral recombinant vector, the lentiviral base vector and a helper plasmid are co-expressed; and
the helper plasmid is selected from the group consisting of SL3, SL4, and SL5.

11. The preparation method according to claim 9, wherein when the recombinant vector is a lentiviral recombinant vector, the lentiviral base vector and a helper plasmid are co-expressed; and
the helper plasmid is selected from the group consisting of SL3, SL4, and SL5.

12. A PML-1 protein and/or a product expressing the PML-1 protein prepared by the preparation method according to claim 8.

13. A PML-1 protein and/or a product expressing the PML-1 protein prepared by the preparation method according to claim 9.

14. A PML-1 protein and/or a product expressing the PML-1 protein prepared by the preparation method according to claim 10.

15. A PML-1 protein and/or a product expressing the PML-1 protein prepared by the preparation method according to claim 11.


SEQUENCE LISTING (17 sequences)

SEQ ID NO: 1 - DNA, 2649 bp, synthetic construct
  source 1..2649: mol_type = other DNA; note = nucleotide sequence encoding PML-1 protein
atggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtcctcccgagagctggatgacagcagcagtgagtccagtgacctccagctggaaggccccagcaccctcagggtcctggacgagaaccttgctgacccccaagcagaagacagacctctggttttctttgacctcaagattgacaatgaaacccagaagattagccagctggctgcggtgaaccgggaaagcaagttccgcgtggtcatccagcctgaagccttcttcagcatctactccaaggccgtgtccctggaggtggggctgcagcacttcctcagctttctgagctccatgcgccgccctatcttggcctgctacaagctgtgggggcctggcctcccaaacttcttccgggccctggaggacattaacaggctgtgggaattccaggaggccatctcgggcttcctggctgccctgcctctcatccgggagcgtgtgcccggggccagcagcttcaaactcaagaacctggcccagacctacctggcgagaaacatgagcgagcgcagcgccatggctgccgtgctggccatgcgtgacctgtgccgcctcctcgaggtctccccgggcccccagctggcccagcatgtctaccccttcagtagcctgcagtgctttgcctccctgcagcccctggtgcaggcagctgtgctgccccgggctgaggcccgcctcctggccctacacaacgtgagcttcatggagctgctgagtgcacaccgccgtgaccggcaggggggcctgaagaagtacagccgctatctaagcctgcagaccaccacgttgccccctgcccagcctgctttcaacctgcaggctctgggcacctactttgaaggcctgttggagggtccggcgctggcacgggcagaaggagtctccaccccacttgctggccgtggcttggcagagagggcctcccagcagagctga

SEQ ID NO: 2 - DNA, 11879 bp, synthetic construct
  source 1..11879: mol_type = other DNA; note = sequence of overexpression vector of the PML-1 gene
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggattgataatcgaattccacgtgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccgccaccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtcctcccgagagctggatgacagcagcagtgagtccagtgacctccagctggaaggccccagcaccctcagggtcctggacgagaaccttgctgacccccaagcagaagacagacctctggttttctttgacctcaagattgacaatgaaacccagaagattagccagctggctgcggtgaaccgggaaagcaagttccgcgtggtcatccagcctgaagccttcttcagcatctactccaaggccgtgtccctggaggtggggctgcagcacttcctcagctttctgagctccatgcgccgccctatcttggcctgctacaagctgtgggggcctggcctcccaaacttcttccgggccctggaggacattaacaggctgtgggaattccaggaggccatctcgggcttcctggctgccctgcctctcatccgggagcgtgtgcccggggccagcagcttcaaactcaagaacctggcccagacctacctggcgagaaacatgagcgagcgcagcgccatggctgccgtgctggccatgcgtgacctgtgccgcctcctcgaggtctccccgggcccccagctggcccagcatgtctaccccttcagtagcctgcagtgctttgcctccctgcagcccctggtgcaggcagctgtgctgccccgggctgaggcccgcctcctggccctacacaacgtgagcttcatggagctgctgagtgcacaccgccgtgaccggcaggggggcctgaagaagtacagccgctatctaagcctgcagaccaccacgttgccccctgcccagcctgctttcaacctgcaggctctgggcacctactttgaaggcctgttggagggtccggcgctggcacgggcagaaggagtctccaccccacttgctggccgtggcttggcagagagggcctcccagcagagctgaggcgcgccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcgatcgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggctccggagagggcaggggaagtcttctaacatgcggggacgtggaggaaaatcccggccccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 3 - protein, 882 aa, Homo sapiens
  source 1..882: note = amino acid sequence of the PML-1 protein
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLPEEAERVKAQVQALGLAEAQPMAVVQSVPGAHPVPVYAFSIKGPSYGEDVSNTTTAQKRKCSQTQCPRKVIKMESEEGKEARLARSSPEQPRPSTSKAVSPPHLDGPPSPRSPVIGSEVFLPNSNHVASGAGEAEERVVVISSSEDSDAENSSSRELDDSSSESSDLQLEGPSTLRVLDENLADPQAEDRPLVFFDLKIDNETQKISQLAAVNRESKFRVVIQPEAFFSIYSKAVSLEVGLQHFLSFLSSMRRPILACYKLWGPGLPNFFRALEDINRLWEFQEAISGFLAALPLIRERVPGASSFKLKNLAQTYLARNMSERSAMAAVLAMRDLCRLLEVSPGPQLAQHVYPFSSLQCFASLQPLVQAAVLPRAEARLLALHNVSFMELLSAHRRDRQGGLKKYSRYLSLQTTTLPPAQPAFNLQALGTYFEGLLEGPALARAEGVSTPLAGRGLAERASQQS

SEQ ID NO: 4 - DNA, 20 bp, synthetic construct
  source 1..20: mol_type = other DNA; note = Sequence of primer SC493-1-P1 for detection of EF1A promoter
actagtgattatcggattga

SEQ ID NO: 5 - DNA, 20 bp, synthetic construct
  source 1..20: mol_type = other DNA; note = Sequence of primer SC493-1-P2 for detection of EF1A promoter
gaagctcgggatcaagaatc

SEQ ID NO: 6 - DNA, 20 bp, synthetic construct
  source 1..20: mol_type = other DNA; note = Sequence of primer SC493-1-P3 for detection of EF1A promoter
cttcatgtgactccacggag

SEQ ID NO: 7 - DNA, 20 bp, synthetic construct
  source 1..20: mol_type = other DNA; note = Sequence of primer SC493-1-P4 for detection of EF1A promoter
caggttggcagcaacgatga

SEQ ID NO: 8 - DNA, 22 bp, synthetic construct
  source 1..22: mol_type = other DNA; note = Sequence of primer SC493-1-P5 for detection of PML-1
aggcatcctgagcacagcgtgt

SEQ ID NO: 9 - DNA, 20 bp, synthetic construct
  source 1..20: mol_type = other DNA; note = Sequence of primer SC493-1-P6 for detection of PML-1
aactcaagaacctggcccag

SEQ ID NO: 10 - DNA, 20 bp, synthetic construct
  source 1..20: mol_type = other DNA; note = Sequence of primer SC493-1-P7 for detection of PML-1
cgtcagcaaacacagtgcac

SEQ ID NO: 11 - DNA, 20 bp, synthetic construct
  source 1..20: mol_type = other DNA; note = Sequence of forward primer hGAPDH-F for qPCR
gagtccactggcgtcttcac

SEQ ID NO: 12 - DNA, 20 bp, synthetic construct
  source 1..20: mol_type = other DNA; note = Sequence of reverse primer hGAPDH-R for qPCR
atggttcacacccatgacga

SEQ ID NO: 13 - DNA, 21 bp, synthetic construct
  source 1..21: mol_type = other DNA; note = Sequence of forward primer hPML-1-R for qPCR
gacttctggtgctttgagtgc

SEQ ID NO: 14 - DNA, 21 bp, synthetic construct
  source 1..21: mol_type = other DNA; note = Sequence of reverse primer hPML-1-R for qPCR
cttggaacatcctcggcagta

SEQ ID NO: 15 - DNA, 2697 bp, synthetic construct
  source 1..2697: mol_type = other DNA; note = nucleotide sequence encoding PML-1 protein
gaattcccgccgccaccatggagccagctccagccagaagccctagacctcagcaggaccccgccagacctcaggaacctaccatgcctcctccagagacacctagcgagggcagacagccttctcctagcccttctcctacagagagagccccagctagcgaggaagagttccagttcctccgctgccagcagtgtcaggcagaagccaagtgccctaagctgctgccttgcctgcacaccctctgcagtggttgtctggaggctagcggcatgcagtgtcccatttgccaggctccttggcctctgggagcagataccccagccctggacaacgtgttcttcgagagcctgcagcggagactgagcgtgtacagacagatcgtggacgctcaggccgtgtgtacacgctgcaaggaatccgccgacttttggtgcttcgagtgcgagcagctcctctgcgctaagtgcttcgaggcccaccagtggttcctgaagcacgaagccagacctctggccgaactgagaaaccagagcgtgcgagagtttctggacggcacccgcaagaccaacaacatcttctgcagcaaccccaaccacagaacccctaccctgaccagcatctattgccggggttgcagcaagcccctgtgttgctcttgcgctctgctggacagcagccacagcgaactcaagtgcgacatcagcgccgagatccagcagagacaggaggaactggacgccatgacacaggctctgcaggaacaggatagcgcctttggagcagtgcacgctcagatgcacgcagcagtgggacagctgggaagagccagagccgaaacagaggagctgatcagggagagagtgagacaggtggtggctcacgtcagagcccaggaaagagagctgctggaggcagtggacgctagataccagcgggactacgaggagatggcctctagactgggcagactggacgccgtgctgcagagaatcaggacaggaagcgccctggtgcagcgcatgaagtgttacgctagcgatcaggaggtgctggacatgcacggattcctgaggcaggctctctgcagactgaggcaggaagagccacagtctctgcaggcagcagtgcggacagacggcttcgacgagttcaaggtccgcctgcaggatctgagcagttgcatcacccagggaaaagacgccgccgtgtctaagaaggcctctccagaagccgcctctacacctagggaccctatcgacgtggatctgccagaggaggcagagagagtgaaggctcaggtgcaggctctgggactggcagaagctcagcctatggcagtggtgcagtcagtgccaggagctcatccagtgcccgtgtacgccttcagcatcaagggacctagctacggcgaggacgtgtccaataccaccaccgcccagaagcgcaagtgttctcagacccagtgcccccggaaggtcatcaagatggagagcgaggagggcaaggaggctagactggctagaagcagcccagagcagcctagacctagcacaagcaaggccgtgtctcctcctcacctggacggacctccttctcctagaagcccagtgatcggcagcgaagtgttcctgcctaatagcaatcacgtggccagcggcgcaggagaagccgaggagagagtggtcgtgatcagcagcagcgaggatagcgacgccgagaatagcagcagcagagagctggacgactctagcagcgagtctagcgacctgcagctggaaggaccttccacactgagggtgctggacgagaacctggcagatcctcaggccgaggatagacctctggtgttcttcgacctgaagatcgacaacgagacccagaagatctctcagctggccgcagtgaaccgggagagcaagttcagggtcgtgatccagcccgaggccttcttcagcatctacagcaaggccgtgtctctggaagtcggactgcagcacttcctgagcttcctgagcagcatgcggaggcccatcctggcttgctacaagctctggggcccaggcctgcctaatttcttcagagccctcgaggacatcaaccgcctctgggagttccaggaggccattagcggatttctggccgctctgcccctgatcagagagagagtgccaggagccagcagcttcaagctgaagaacctggcccagacctacctggctcggaacatgagcgagagaagcgctatggcagccgtgctggctatgagagacctgtgcaggctgctggaggtgtcaccaggacctcagctggctcagcacgtgtatcccttcagcagcctccagtgtttcgcctctctgcagcctctggtgcaggcagcagtgctgcctagagcagaagctagactgctggccctgcacaacgtgtccttcatggagctgctgagcgcccatagaagagacagacagggaggcctgaagaagtacagccgctacctgagcctgcagacaacaacactgcctccagcccagccagctttcaacctgcaggctctgggcacctacttcgaaggactgctggaaggaccagctctggctagagcagagggagtgtctacacctctggccggaagaggactggcagaaagagccagccagcagtctcaccaccaccatcaccaccaccactgatggatcc

SEQ ID NO: 16 - DNA, 11 bp, synthetic construct
  source 1..11: mol_type = other DNA; note = base fragment inserted before the sequence of GAATTC in the original pcDNA3.4 vector
ggatctctagc

SEQ ID NO: 17 - DNA, 12 bp, synthetic construct
  source 1..12: mol_type = other DNA; note = base fragment inserted after the sequence of GGATCC of the original pcDNA3.4 vector
cccgacctcgac